(19) United States
(12) Patent Application Publication
(10) Pub. No.: US 2006/0024699 A1
(43) Pub. Date: Feb. 2, 2006

(54) Method for evaluating a substance capable of effecting on endoplasmic reticulum stress-and/or amyloid beta-induced apoptosis

(76) Inventors: Junichi Hitomi, Osaka (JP); Taiichi Katayama, Osaka (JP); Yutaka Eguchi, Osaka (JP); Yoshihide Tsujimoto, Osaka (JP); Masaya Tohyama, Osaka (JP)

Correspondence Address: BIRCH STEWART KOLASCH & BIRCH, PO BOX 747, FALLS CHURCH, VA, 22040-0747, US

(21) Appl. No.: 11/124,103

(22) Filed: May 9, 2005

(30) Foreign Application Priority Data
Jun. 23, 2004 (JP) 2004-184516

(51) Int. Cl. C12Q 1/68; G01N 33/574

(52) U.S. Cl. 435/6; 435/7.23

(57) ABSTRACT

The present invention relates to a method for evaluating a substance capable of affecting endoplasmic reticulum stress-induced apoptosis and/or amyloid-β-induced apoptosis, a kit for evaluating the substance and a pharmaceutical composition.

### BACKGROUND OF THE INVENTION

1. Field of the Invention

The present invention relates to a method for evaluating a substance capable of affecting endoplasmic reticulum stress-induced apoptosis and/or amyloid-β-induced apoptosis, a system for evaluating the substance, and a pharmaceutical composition capable of inhibiting a disease or progress thereof caused in association with the endoplasmic reticulum stress-induced apoptosis and/or amyloid-β-induced apoptosis.

2. Discussion of Related Art

It is known that apoptotic cell death in a living body is found in physical events such as developmental process or replacement of normal cells and in pathological events such as viral infection. It has been shown that such apoptotic cell death is also induced by, for example, ultraviolet, radiation, excess or long-term endoplasmic reticulum stress, and the like.

It has been reported that the unfolded protein response increases the transcription of CHOP, which is closely associated with cell death (Brewer J. W. et al., “A pathway distinct from the mammalian unfolded protein response regulates expression of endoplasmic reticulum chaperones in non-stressed cells.”, EMBO J., 16: 7207-7216 (1997)). It has also been reported that recruitment of TNF receptor-associated factor 2 (TRAF2) to activated IRE1α induces c-Jun N-terminal kinase (JNK) activation (Urano, F. et al., “Coupling of stress in the ER to activation of JNK protein kinases by transmembrane protein kinase IRE1.”, Science, 287, 664-666 (2000)). Furthermore, it has been reported that calpain activates downstream caspase-cascade (Nakagawa et al, “Cross-talk between two cysteine protease families. Activation of caspase-12 by calpain in apoptosis.”, J. Cell Biol., 150: 887-894 (2000)).

It is also believed that activation of caspases, a family of cysteine proteases that cleave substrates at specific aspartate residues, is a central mechanism in the apoptotic cell death process (see Salvesen, G. S. et al, “Caspases: intracellular signaling by proteolysis.”, Cell, 91: 443-446 (1997); and Thornberry, N. et al, “Caspases: enemies within.”, Science, 281: 1312-1316 (1998)).

Among the known caspases, mouse caspase-12 seems to be involved in signaling pathways specific to endoplasmic reticulum stress-induced apoptosis (see Nakagawa, T. et al., “Caspase-12 mediates endoplasmic-reticulum-specific apoptosis and cytotoxicity by amyloid-β.”, Nature, 403: 98-103 (2000)).

On the other hand, regarding human caspase-12 corresponding to the mouse caspase-12, existence of a sequence that is highly homologous to a nucleic acid encoding the mouse caspase-12 has been found at the locus within the caspase-1/ICE (interleukin-1β converting enzyme) gene cluster on human chromosome 11q22.3. However, the sequence is interrupted by frame shift and premature stop codon, and also has amino acid substitution in the critical site for caspase activity. Therefore, it has been suggested that a nucleic acid having the sequence or a product thereof does not fulfill a function (see Fischer H. et al., “Human caspase 12 has acquired deleterious mutations.”, Biochem. Biophys. Res. Commun. 293: 722-726 (2002)).

It has also been suggested that caspase-12 in human is only found in the form of an incomplete polypeptide in Caucasian race and Asian race, and that a polymorphism of full-length caspase-12 is expressed and not involved in endoplasmic reticulum stress-mediated apoptosis in Black race (see Maya Saleh et al, “Differential modulation of endotoxin responsiveness by human caspase-12 polymorphism”, Nature, 429, 75-79 (2004)).

### SUMMARY OF THE INVENTION

It is an aspect of the present invention to provide a method for evaluating a substance capable of affecting endoplasmic reticulum stress-induced and/or amyloid-β-induced apoptosis, which enables at least one of matters including carrying out screening and/or validation of a substance which affects (for example, suppress or enhance) apoptosis based on a pathway different from apoptotic signaling pathways in mitochondria, especially a substance which specifically affects endoplasmic reticulum stress-induced and/or amyloid-β-induced apoptosis, carrying out screening and/or validation of a substance capable of inhibiting a disease or progress thereof caused in association with endoplasmic reticulum stress-induced and/or amyloid-β-induced apoptosis, and developing means for treating or preventing a disease caused by endoplasmic reticulum stress-induced apoptosis and/or amyloid-β-induced apoptosis. It is another aspect of the present invention to provide a system for evaluating a substance capable of affecting endoplasmic reticulum stress-induced and/or amyloid-β-induced apoptosis, for example, a kit for evaluation, which enables at least one of matters including, rapidly, easily, well-repeatably, or accurately carrying out the evaluation method, and carrying out the evaluation method with more reflecting the conditions in a living body. It is still another aspect of the present invention to provide a pharmaceutical composition which enables to inhibit a disease or progress thereof caused by endoplasmic reticulum stress-induced and/or amyloid-β-induced apoptosis. Here, the further problems are clear from the present specification.

Specifically, the gist of the present invention relates to
1. a method for evaluating a substance capable of affecting endoplasmic reticulum stress-induced apoptosis and/or amyloid-β-induced apoptosis, which comprises the step of contacting a substance to be tested with a cell expressing pro-caspase-4, thereby examining behaviors mediated by caspase-4 in the cell in the presence of a substance causing endoplasmic reticulum stress or amyloid-β;
2. the method according to the above item 1, wherein the cell expressing pro-caspase-4 is a cell selected from the group consisting of SK-N-SH cell, HeLa cell, HepG2 cell, SY-SY cell and a transfected cell into which a nucleic acid encoding pro-caspase-4 is introduced;
3. the evaluation method according to the above item 1, wherein the cell expressing pro-caspase-4 is a transfected cell obtained by introducing a nucleic acid encoding pro-caspase-4 into HEK293 cell or HEK 293T cell;
4. the evaluation method according to the above item 1, which comprises the steps of:
   (1) contacting a substance to be tested with a cell expressing pro-caspase-4,
   (2) culturing the cell obtained in the above step (1) in the presence of a substance causing endoplasmic reticulum stress or amyloid-β, and
   (3) examining change specific to apoptosis, for the cells obtained after the above step (2);
5. the evaluation method according to the above item 1, which comprises the steps of:
   (A) culturing a cell expressing pro-caspase-4, in the presence of a substance to be tested and a substance causing endoplasmic reticulum stress or amyloid-β, and
   (B) examining changes specific to apoptosis, for the cell obtained after carrying out the above step (A);
6. the evaluation method according to the above item 1, wherein the presence or absence of changes in behavior caused by the presence of a substance to be tested is determined in the presence of a substrate of caspase-4;
7. the evaluation method according to the above item 1, wherein the substance to be tested is a substance obtained by carrying out the steps of:
   a) screening a substance binding to pro-caspase-4,
   b) contacting the substance obtained in the above step a) with pro-caspase-4 in the presence of a substance causing endoplasmic reticulum stress or amyloid-β, to thereby screen a substance generating cleaved products thereof, and
   c) preparing a substance to be tested, on the basis of a cleavage site of the substance obtained in the above step b) by caspase-4, as a derivative containing the cleavage site region of the substance;
8. the evaluation method according to the above item 1, wherein the substance to be tested is a substance selected from the group consisting of a derivative of a substrate of caspase-4, a mimic of the substrate and a substrate capable of binding to the caspase-4;
9. the evaluation method according to the above item 1, wherein the behavior is at least one of the events selected from the group consisting of karyopyknosis, fragmentation of a chromosome, shrinkage of a cell, release of MTS, cleavage of pro-caspase-4, release of a lactate dehydrogenase and generation of a product resulted from a substrate of caspase-4,
10. a kit for evaluating a substance capable of affecting endoplasmic reticulum stress-induced apoptosis and/or amyloid-β-induced apoptosis, for carrying out the evaluation method of any one of the above items 1. to 9., which comprises a cell expressing pro-caspase-4, a substance causing endoplasmic reticulum stress or amyloid-β, and a reagent suitable for contacting a substance to be tested with the cell;
11. a pharmaceutical composition, which comprises as an active ingredient, a substance evaluated by the method for evaluation of any one of the above items 1. to 9., wherein the substance is a substance capable of affecting endoplasmic reticulum stress-induced apoptosis and/or amyloid-β-induced apoptosis; and
12. the pharmaceutical composition according to the above item 11, wherein the substance is an siRNA specific to pro-caspase-4 or caspase-4, or an antisence nucleic acid against a nucleic acid encoding caspase-4.

### BRIEF DESCRIPTION OF THE DRAWINGS

FIG. 1 illustrates localization of caspase-4 in SK-N-SH cells and HeLa cells. Panels “a” to “f” show SK-N-SH cells, and Panels “g” to “l” show HeLa cells. Panel “a” and Panel “g” respectively show the staining with an anti-caspase-4 antibody, and Panel “b” and Panel “h” respectively show the staining with an anti-KDEL antibody. Panel “c” and Panel “i” respectively show results displayed by overlapping the results of the staining with an anti-caspase-4 antibody with the results of the staining with an anti-KDEL antibody. Furthermore, Panel “d” and Panel “j” respectively show the staining with an anti-caspase-4 antibody, and Panel “e” and Panel “k” respectively show the staining with trade name: Mitotracker. Panel “f” and Panel “I” respectively show results displayed by overlapping the results of the staining with anti-caspase-4 antibody with the results of the staining with trade name: Mitotracker. Panels “m” to “o” respectively show HeLa cells transfected with a caspase-4-GFP fusion gene. Panel “m” shows the staining with anti-caspase-4 antibody, and Panel “n” shows the staining with trade name: ER-tracker. Panel “o” shows a result displayed by overlapping the result of the staining with an anti-caspase-4 antibody with the result of the staining with trade name: ER-tracker. In FIG. 1, the scale bar represents 5 μm.

FIG. 2 illustrates specific cleavage of caspase-4 by endoplasmic reticulum stress and Aμ treatment. FIG. 2A shows the results of treatment of SK-N-SH cells with 1 μg/ml tunicamycin (TM), 0.5 μM thapsigargin (TG), 100 μM etoposide (Etop), or 0.1 μM staurosporine (STS) for indicated periods, and subsequent incubation thereof for indicated periods or shows the results of irradiation of 150 J/m² UV and subsequent incubation for indicated periods. The FIG. 2A is the results of analysis by Western blotting using various kinds of antibodies. In the above-mentioned FIG. 2A, the upper panel shows an anti-caspase-4 antibody, the middle panel being an anti-caspase-3 antibody and an anti-caspase-7 antibody, and the bottom panel being an anti-β-actin antibody. The top of the gel in FIG. 2A shows extent of cell death assessed by MTS assay after incubation for indicated periods. FIG. 2B shows the results of treatment of SK-N-SH cells with 25 μM Aβ₂₅₋₃₅ or 5 μM Aβ₁₋₄₀ peptides for the indicated periods. The FIG. 2B is the results of analysis by Western blotting using various kinds of antibodies. In the FIG. 2B, the upper panel shows an anti-caspase-4 antibody, and the bottom panel shows an anti-β-actin antibody. FIG. 2C shows the results of treatment of SK-N-SH cells with the peptides of which direction of the amino acid sequence is reversed {Aβ₃₅₋₂₅ (25 μM) and Aβ₄₀₋₁ (5 μM)} for the indicated periods, and subsequent test on the cleavage of caspase-4.

FIG. 3 shows the results of study on the effect of Bcl-2 overexpression on endoplasmic reticulum stress-induced cleavage of caspase-4. Panel “a” of FIG. 3A shows an SK-N-SH cell stably transfected with a vector or a Bcl-2 expression system, Panel “b” of FIG. 3A shows a HeLa cell transfected with the vector or a Bcl-2 expression system. In addition, in Panel “a” of FIG. 3A and Panel “b” of FIG. 3A, “+” represents the results with 1 μg/ml tunicamycin, and “−” represents the results without tunicamycin. Furthermore, the Panel “a” of FIG. 3A and Panel “b” of FIG. 3A show the results of Western blotting using various kinds of antibodies. In the Panel “a” of FIG. 3A and Panel “b” of FIG. 3A, each of the upper panels shows an anti-caspase-4 antibody, and the bottom panels show an anti-Bcl-2 antibody. FIG. 3B shows the results obtained by treating indicated cells with tunicamycin (1 μg/ml) for 30 hours, staining the resulting cells with Hoechst 33342, and observing the resulting cells under a fluorescence microscope. The scale bar represents 25 μm.

FIG. 4 illustrates decrease in endoplasmic reticulum stress-induced cell death or Aβ-induced cell death after reduction of expression of caspase-4 by siRNA. FIG. 4A shows the results of decrease in caspase-4 by introducing caspase against GFP (control) (in the figure, “GFP siRNA-a”) or siRNA against caspase (in the figure, “caspase siRNA”) (1 μg/24 well plate for each). The scale bar represents 5 μm. FIG. 4B illustrates caspase-4 upon inducing endoplasmic reticulum stress in cells transfected with the above-mentioned siRNA. In FIG. 4B, “+” represents the results with 0.5 μM thapsigargin, and “−” represents the results without thapsigargin. In addition, FIG. 4B illustrates the results of Western blotting using various kinds of antibodies. In FIG. 4B, the upper panel shows an anti-caspase-4 antibody, and the lower panel shows an anti-β-actin antibody. Panel “a” of FIG. 4C shows representative phase contrast microscopic images of cells transfected with GFP siRNA after 40 hours of treatment with 0.5 μM thapsigargin, and Panel “b” of FIG. 4C shows representative phase contrast microscopic images of cells transfected with caspase-4 siRNA. Panel “c” of FIG. 4C represents the extent of cell death expressed as the mean ±SEM from three independent experiments. The scale bar represents 50 μm. FIG. 4D and FIG. 4E show cell viability after treatment of cells transfected with each of the siRNAs with 0.5 μM thapsigargin or 100 μM etoposide. FIG. 4D shows SK-N-SH cells, and FIG. 4E shows HeLa cells. The results are expressed as the mean ±SEM for three independent experiments. FIG. 4F shows cell viability when cell death was induced by Aμ in SK-N-SH cells transfected with each of the siRNAs. Each value represents the result expressed as the mean ±SEM for three independent experiments.

FIG. 5 illustrates the results of study on expression of caspase-4 in brains. In the figure, “AD” represents the results of brain tissues from patients with Alzheimer's disease, and “DC” represents the results of brain tissues from patients with other neurodegenerative disorders. The scale bar represents 9.52 μm.

### DETAILED DESCRIPTION OF THE INVENTION

The present invention is based on surprising findings of the present inventors that endoplasmic reticulum stress and/or amyloid-β cleaves pro-caspase-4, to produce caspase-4. Furthermore, the present invention is based on surprising findings that caspase-4 unexpectedly specifically affects endoplasmic reticulum stress-induced and/or amyloid-β-induced apoptosis, whereas the sequence encoding the caspase-4 is not a sequence localized on a locus within the caspase-1/ICE (interleukin-1β converting enzyme) gene cluster on chromosome 11q22.3 which is thought to be corresponding to mouse caspase-12 specifically affecting endoplasmic reticulum stress-induced apoptosis in mouse.

An aspect of the present invention relates to a method for evaluating a substance capable of affecting endoplasmic reticulum stress-induced apoptosis and/or amyloid-β-induced apoptosis, which comprises contacting a substance to be tested with a cell expressing pro-caspase-4, thereby examining behavior mediated by caspase-4 in the cell, in the presence of a substance causing endoplasmic reticulum stress (also referred to as an endoplasmic reticulum stress inducer) or amyloid-β.

The behavior mediated by caspase-4 in a cell is examined in the evaluation method of the present invention. Therefore, according to the evaluation method of the present invention, there is exhibited an excellent effect that a substance specifically affecting endoplasmic reticulum stress-induced and/or amyloid-β-induced apoptosis can be screened rapidly by a simple procedure. The evaluation method of the present invention can be also used for validation of pharmacological evaluation and the like.

The behavior mediated by caspase-4 in a cell is examined in the evaluation method of the present invention. Therefore, according to the evaluation method of the present invention, screening and/or validation of a substance capable of inhibiting specifically a disease or progress thereof caused in association with endoplasmic reticulum stress-induced and/or amyloid-β-induced apoptosis can also be carried out.

Furthermore, since the behavior mediated by the caspase-4 in a cell is examined in the evaluation method of the present invention, according to the present invention, the events caused by caspase-4 can be evaluated with more reflecting the conditions in a living body.

Therefore, according to the evaluation method of the present invention, means for treating or preventing the above-mentioned disease can be developed.

The caspase-4 used in the present invention is caspase-4 from a mammal. When used for treatment of a disease in human, preferably, it is desirable that the caspase-4 be human caspase-4. The nucleic acid encoding human pro-caspase-4 includes (A) a nucleic acid containing the nucleotide sequence shown in SEQ ID NO: 1. In addition, human pro-caspase-4 includes (a) a polypeptide containing the amino acid sequence shown in SEQ ID NO: 2. The nucleic acid encoding human caspase-4 includes (I) a nucleic acid containing a sequence consisting of the sequence shown in the base numbers: 318-1173 of SEQ ID NO: 1 and the like. Human caspase-4 includes (i) a polypeptide containing an amino acid sequence consisting of the sequence shown in the amino acid numbers: 94-377 of SEQ ID NO: 2 and the like.

Here, although mutation is naturally occurring in the nucleotide sequence or in the amino acid sequence shown in the above-mentioned SEQ ID NOs in a living body depending on the kind of an individual, localizing tissues and the like, there are cases where a nucleic acid (translational product thereof) consisting of a nucleotide sequence having the mutation or a polypeptide consisting of an amino acid sequence having the mutation exhibits a functional activity. Therefore, the nucleic acid encoding pro-caspase-4, the pro-caspase-4, the nucleic acid encoding caspase-4 and the caspase-4 which are used in the present invention include variants of each of (A), (a), (I) and (i), as long as they have functional activities.

Here, in the present specification, the term “functional activity” means, when used in association with pro-caspase-4, a functional activity to generate cleavage product having cysteine protease activity by endoplasmic reticulum stress and/or amyloid-β, and the term means, when used in association with caspase-4, cysteine protease activity. In addition, in the present specification, “functional activity” regarding the nucleic acid also includes an activity expressed via a polypeptide encoded by the nucleic acid (i.e., translational product).

As for a variant of pro-caspase-4, the functional activity is evaluated by
introducing a nucleic acid encoding a variant to be tested into a cell and expressing the variant in the cell, and
maintaining the cell in the presence of endoplasmic reticulum stress and/or amyloid-β.
   Here, cleavage of a variant of pro-caspase-4 to produce a cleavage product exhibiting cysteine protease activity is an index to indicate that the variant has the functional activity.

As for a variant of caspase-4, the functional activity is evaluated by introducing a nucleic acid encoding a variant to be tested into a cell and expressing the variant in the cell. Here, cysteine protease activity exhibited by the variant is an index indicating that the variant has the functional activity. The cysteine protease activity is, for example, determined using Ac-WEHD-AMC in which a fluorescent dye 7-amino-4-methylcoumarin (AMC) is covalently bound to a tetrapeptide WHED (Trp-His-Glu-Asp; SEQ ID NO: 9) which is specifically recognized by caspase-4 as a substrate. Specifically, the cysteine protease activity can be determined by quantifying AMC which is released by cleaving the substrate by cysteine protease activity of caspase-4. Here, for such determination of cysteine protease activity, for example, a reaction solution having a composition: 0.1 M HEPES, pH 7.0, 10% by weight of polyethylene glycol, 0.1% by volume of 3-((3-cholamidopropyl)-dimethylammonio)-1-propane sulfonate (CHAPS), 10 mM dithiothreitol, pH7.0; and the like can be used. Here, in the present specification, 1U of the cysteine protease activity is defined as the amount of an enzyme needed for production of 1 pmol AMC/min. when a saturation concentration of substance at room temperature (25° C.) is used.

The variant of a nucleic acid encoding pro-caspase-4 includes:
(B) a nucleic acid containing a sequence having a mutation (substitution, deletion, addition or insertion) in at least one nucleotide residue in the sequence shown in SEQ ID NO: 1, wherein a polypeptide encoded by the sequence is a polypeptide capable of producing a cleaved product having cysteine protease activity by endoplasmic reticulum stress and/or amyloid-β,
(C) a nucleic acid containing a sequence of a nucleic acid capable of hybridizing with an antisense strand of a nucleic acid consisting of the sequence shown in SEQ ID NO: 1 under stringent conditions, wherein a polypeptide encoded by the sequence is a polypeptide capable of producing a cleaved product having cysteine protease activity by endoplasmic reticulum stress and/or amyloid-β,
(D) a nucleic acid of which sequence is a sequence having sequence identity of at least 90% with the sequence shown in SEQ ID NO: 1, wherein a polypeptide encoded by the sequence is a polypeptide capable of producing a cleaved product having cysteine protease activity by endoplasmic reticulum stress and/or amyloid-β,
(E) a nucleic acid different from the nucleic acid according to the above item (A) via degeneracy, and the like.

The variant of pro-caspase-4 includes:
(b) a polypeptide containing an amino acid sequence having a mutation (substitution, deletion, addition or insertion) in at least one amino acid residue in the sequence shown in SEQ ID NO: 2, wherein the polypeptide is a polypeptide capable of producing a cleaved product having cysteine protease activity by endoplasmic reticulum stress and/or amyloid-β,
(c) a polypeptide which is encoded by a nucleic acid capable of hybridizing with an antisense strand of a nucleic acid consisting of the sequence shown in SEQ ID NO: 1 under stringent conditions, wherein the polypeptide is a polypeptide capable of producing a cleaved product having cysteine protease activity by endoplasmic reticulum stress and/or amyloid-β,
(d) a polypeptide containing an amino acid sequence having sequence identity of at least 90% with the sequence shown in SEQ ID NO: 2, wherein the polypeptide is a polypeptide capable of producing a cleaved product having cysteine protease activity by endoplasmic reticulum stress and/or amyloid-P.

The variant of a nucleic acid encoding caspase-4 includes:
(II) a. nucleic acid containing a nucleotide sequence having a mutation (substitution, deletion, addition or insertion) in at least one nucleotide residue in the sequence shown in the base numbers: 318-1173 of SEQ ID NO: 1, wherein a polypeptide encoded by the nucleotide sequence is a polypeptide having cysteine protease activity,
(III) a nucleic acid containing a nucleotide sequence of a nucleic acid capable of hybridizing with an antisense strand of a nucleic acid consisting of a sequence shown in the base numbers: 318-1173 of SEQ ID NO: 1 under stringent conditions, wherein a polypeptide encoded by the nucleotide sequence is a polypeptide having cysteine protease activity,
(IV) a nucleic acid of which nucleotide sequence is a sequence having sequence identity of at least 90% with the sequence shown in the base numbers: 318-1173 of SEQ ID NO: 1, wherein a polypeptide encoded by the sequence is a polypeptide having cysteine protease activity,
(V) a nucleic acid different from the nucleic acid of the above item (I) via degeneracy, and the like. Here, it is understood that the above pro-caspase-4 (A) and its variants (B) to (D) are not encompassed in the variants of caspase-4 (II) to (V).

The variant of caspase-4 includes:
(ii) a polypeptide containing an amino acid sequence having a mutation (substitution, deletion, addition or insertion) in at least one amino acid residue in the sequence shown in the amino acid numbers: 94-377 of SEQ ID NO: 2, wherein the polypeptide has cysteine protease activity,
(iii) a polypeptide which is encoded by a nucleic acid capable of hybridizing with an antisense strand of a nucleic acid consisting of the sequence shown in the base numbers: 318-1173 of SEQ ID NO: 1 under stringent conditions, wherein the polypeptide has cysteine protease activity,
(iv) a polypeptide which is encoded by a nucleic acid capable of hybridizing with an antisense strand of a nucleic acid consisting of a sequence corresponding to the amino acid numbers: 94-377 of SEQ ID NO: 2 under stringent conditions, wherein the polypeptide has cysteine protease activity,
(v) a polypeptide containing an amino acid sequence having sequence identity of at least 90% with the sequence shown in SEQ ID NO: 2, wherein the polypeptide has cysteine protease activity, and the like. Here, it is understood that the above pro-caspase-4 (a) and its variants (b) to (d) are not encompassed in the variants of caspase-4 (ii) to (v).

In the present specification, “at least one nucleotide residue” may be the number of nucleotide residues within a range so that an encoded polypeptide has a functional activity (i.e., a range so that the polypeptide exhibits a functional activity). Specifically, “at least one nucleotide residue” refers to, for example, one or plural, preferably one or several amino acid residues.

The nucleic acid of the above item (B) or (II) or the polypeptide of the above item (b) or (ii) can be obtained by
obtaining a candidate nucleic acid by introduction of a mutation into a nucleic acid consisting of the sequence shown in SEQ ID NO: 1 or the sequence shown in the base numbers: 318-1173 of SEQ ID NO: 1 by conventional site-directed mutagenesis, synthesis of a nucleic acid having a mutation referring to the above sequence, or the like,
incorporating the resulting nucleic acid into a suitable vector and expressing a polypeptide encoded by the nucleic acid in an appropriate cell, and
evaluating a functional activity as described above, and selecting a nucleic acid (i.e., its expression product) or a polypeptide having the functional activity.

The vector can be appropriately selected depending on the kind of a cell to be used and the like. The vector includes, for example, a plasmid vector, a virus vector and the like. Such vector can be appropriately selected depending on the cell to be used. In addition, the vector may be any of an intracellular direct expression type vector, a secretion expression type vector and a fusion protein expression type vector. Specifically, the vector includes pKCR, pEFBOS, cDM8, pCEV4, pcDNA3.1, pcDNA3, pcDNA4, pcDNA6, pSFV, pCAGSS and the like. Such vector may appropriately have a factor such as an inducible promoter, a marker gene for selection, terminator. In addition, the vector may appropriately contain a tag sequence and the like for expression as a fusion protein. Instead of using the vector, a construct obtained by making a carrier such as liposome or DEAE-dextran to carry a nucleic acid to be introduced may be used.

The cell includes, for example, SK-N-SH cell, HeLa cell, SY-SY cell, HEK293 cell, HEK293T cell and the like.

Here, introduction of a nucleic acid into a cell can be carried out by a conventional method for introducing a gene, such as electroporation method, DEAE-dextran method, calcium-phosphate method, lipofection method, transfection method, method for introduction by a particle gun.

In the present specification, “stringent conditions” include such conditions described in Molecular Cloning: A Laboratory Manual 3^{rd} Ed., Cold Spring Harbor Laboratory Press (2001), all teachings of which are incorporated herein by reference, and the like. From the viewpoint of enhancing probability of a nucleic acid or a polypeptide having a functional activity, it is desirable that the “stringent conditions” is preferably medium stringent conditions, more preferably high stringent conditions. More specifically, the stringent conditions include, for example, conditions so that incubation is carried out in a solution containing 20×SSC (composition of 1×SSC: 0.15 M NaCl, 0.015 M sodium citrate, pH 7.0), 0.1% SDS, 5× Denhart and 100 mg/ml herring sperm DNA at 65° C. overnight, and washing is carried out with a solution containing 2×SSC and 0.1% SDS at room temperature for 15 minutes.

In addition, in the hybridization under such stringent conditions, from the viewpoint of further enhancing accuracy, incubation is carried out under lower-ion strength conditions, for example, conditions such as 5×SSC, more stringently 3×SSC, and/or higher-temperature conditions, for example, 25° C. lower, more stringently 22° C. lower, even more stringently 20° C. lower than the Tm value of a nucleic acid to be used, specifically, though varying depending on the Tm value of a nucleic acid to be used, 65° C. or higher, more stringently 67° C. or higher, even more stringently 70° C. or higher. From the same viewpoint, washing and the like under more stringent washing conditions, specifically, using a buffer having lower ion strength, for example 2×SSC, more stringently 1×SSC, even more stringently 0.5×SSC and the like, under the conditions, for example, 40° C. lower, more stringently 30° C. lower, even more stringently 25° C. lower than the Tm value of a nucleic acid to be used, specifically, though varying depending on the Tm value of a nucleic acid to be used, 30° C. or higher, more stringently 37° C. or higher, even more stringently 42° C. or higher, even further stringently 45° C. or higher and the like. Such hybridization enables, for example, to obtain a nucleic acid having sequence identity of at least 90%, preferably 90% or more, more preferably 97% or more with the sequence shown in SEQ ID NO: 1. Here, Tm can be calculated according to the description in the above-mentioned Molecular Cloning: A Laboratory Manual 3^{rd} Ed., and the like.

The nucleic acid of the above item (C) or (III) or a polypeptide of the above item (c) or (iii) can be obtained by,
carrying out hybridization of a nucleic acid consisting of the nucleotide sequence shown in SEQ ID NO: 1 or the nucleotide sequence shown in the base numbers: 318-1173 of the SEQ ID NO: 1 with a nucleic acid to be tested under the stringent conditions,
incorporating the resulting nucleic acid into an appropriate vector and then expressing in an appropriate cell,
evaluating a functional activity as described above, to select a nucleic acid or a polypeptide having the functional activity. In addition, the polypeptide of the above item (iv) can be obtained in the same procedure as preparation of the nucleic acid of the above item (C) or (III) or a polypeptide of the above item (c) or (iii), using a nucleic acid consisting of a sequence corresponding to the amino acid numbers: 94-377 of SEQ ID NO: 2.

Here, a vector, a cell and a method for introducing a nucleic acid into a cell are same as described above. In addition, the hybridization can be carried out by the method described in the above-mentioned Molecular Cloning: A Laboratory Manual 3^{rd} Ed., and the like.

Here, in the present specification, it is desirable that the “nucleic acid capable of hybridizing with an antisense strand of a nucleic acid consisting of the sequence shown in the base numbers: 318-1173 of SEQ ID NO: 1 under stringent conditions” is preferably a nucleic acid consisting of a sequence having a length substantially equivalent to that of a nucleic acid consisting of the sequence shown in the base numbers: 318-1173 of the SEQ ID NO: 1. In addition, it is desirable that the “nucleic acid capable of hybridizing with an antisense strand of a nucleic acid consisting of a sequence corresponding to the amino acid numbers: 94-377 of SEQ ID NO: 2 under stringent conditions” is preferably a nucleic acid consisting of a sequence having a length substantially equivalent to that of a nucleic acid consisting of a sequence corresponding to the amino acid numbers: 94-377 of the SEQ ID NO: 2. Here, “a length substantially equivalent” means that the difference from a specific sequence is 0 to 400 nucleotides in length, preferably 0 to 200 nucleotides in length, more preferably 0 to 100 nucleotides in length.

In the present specification, sequence identity refers to a value calculated by properly aligning a nucleotide sequence with a reference sequence under a parameter value of Cost to open gap 5, Cost to extend gap 2, Penalty for nucleotide mismatch -3, reward nucleotide match 1, expect value 10, wordsize 11, gap existence 10, gap extension 1, or aligning an amino acid sequence with a reference sequence under a parameter value of Cost to open gap 5, Cost to extend gap 2, Penalty for nucleotide mismatch -3, reward nucleotide match 1, expect value 10, wordsize 11, gap existence 10, gap extension 1, on BLAST algorism, for example, determining the identical residue present in the both sequence to determine the number of matched sites, subsequently dividing the above number of the matched sites by the total number of the residues within the sequence region to be compared, and multiplying the resulting value by 100. Here, the BLAST algorism is available via the home page of National Institutes of Health.

The substance to be tested to which the evaluation method of the present invention can be applied includes, for example, a derivative of a substrate of caspase-4, a mimic of the substrate, a substrate capable of binding to the caspase-4 and the like. The substance to be tested includes, specifically, a compound, a peptide, a peptide mimic, various kinds of proteins, a nucleic acid, a nucleic acid analog, an antibody, a fragment of an antibody and the like.

The compound can be a compound of a combinatorial library obtained by combinatorial chemistry.

The nucleic acid and the nucleic acid analog include a sense nucleic acid, an antisense nucleic acid, ribozyme, siRNA and the like. Here, especially, from the viewpoint of improving stability of DNA, a PNA having a peptide skeleton can be employed.

The nucleic acid and the nucleic acid analog can be designed by referring to a database of known sequences, on the basis of a sequence having low sequence identity to the known sequences, for example, a sequence having sequence identity of preferably 15% or less, more preferably 10% or less, even more preferably 5% or less, even further more preferably 0%, wherein the sequence identity is calculated by aligning appropriately the sequence to a reference sequence under the same parameter values as described above on the basis of BLAST algorism.

The strand length of the nucleic acid and the nucleic acid analog can be appropriately defined depending on the kind of the nucleic acid and the nucleic acid analog.

The strand length of a sense nucleic acid or an antisense nucleic acid is not particularly limited, but can be defined appropriately.

From the viewpoint of lowering the effect of binding factor irrelevant to RNAi, it is desirable that the sequence of the siRNA is a sequence without 5′untranslated region, 3′untranslated region, a region around an initiating codon and the like. The sequence of the siRNA can be designed by, for example, a conventional siRNA design tool (for example, siRNA design tool manufactured by Dharmacon, and the like).

It is desirable that the siRNA is, but not particularly limited to, a sequence corresponding to a region at preferably 50 to 100 nucleotides or more, more preferably at least 75 nucleotides downstream from an initiating codon of a target sequence of which function is to be inhibited in a nucleic acid encoding pro-caspase-4 or caspase-4. In addition, it is desirable that the siRNA is a sequence consisting of two contiguous adenylic acid residues and 19 arbitrary nucleotide residues or a sequence of two contiguous adenylic acid residues and 21 arbitrary nucleotide residues, more preferably a sequence consisting of two adenylic acid residues, one guanylic acid residue and 18 arbitrary nucleic acid residues or a sequence consisting of two adenylic acid residues, one cytidylic acid residue and 18 arbitrary nucleic residues. Furthermore, it is desirable that the GC content of the siRNA is preferably 30% to 70%, more preferably around 40% to around 50%, even more preferably around 50%. It is desirable that such siRNA is a sequence specific for a target sequence having low sequence identity to the sequences in a database of known sequences.

In addition, it is desirable, for example in the case of a tRNA-linked ribozyme, that the ribozyme is a ribozyme complementary to the recognition sequence having a sequence which can be cleaved by a ribozyme in a target sequence for inhibiting the function of a nucleic acid encoding pro-caspase-4 or caspase-4 and adjacent 6 to 9 nucleotides on each side. It is also desirable that the ribozyme is, for example, a nucleic acid which accurately forms a conformation of a tRNA and has a stable conformation, and which does not form a stem conformation in a mRNA to be targeted.

Here, the substance to be tested can be a substance obtained by carrying out the steps of:
a) screening a substance binding to pro-caspase-4 or caspase-4,
b) contacting the substance obtained in the above step a) with caspase-4, in the presence of a substance causing endoplasmic reticulum stress or amyloid-β, to give a product, and
c) preparing a substance to be tested, on the basis of a cleavage site of the product obtained in the above step b) cleaved by caspase-4, as a derivative containing cleavage site region of the product.

In the step a), screening of a substance binding to pro-caspase-4 or caspase-4 can be carried out by two-hybrid method, co-immunoprecipitation method, pull-down method, plasmon resonance interaction analysis and the like.

The above-mentioned two-hybrid method is carried out by, for example,
1) incorporating a nucleic acid encoding pro-caspase-4 or caspase-4 into a prey vector, to give a prey plasmid,
2) introducing the resulting prey plasmid into an yeast (EGY48 (p8op lacZ)), to give a prey plasmid-carrying clone,
3) incorporating a nucleic acid encoding a substance to be tested (polypeptide) into a bait vector, to give a bait plasmid,
4) introducing the bait plasmid obtained in step 3) into the prey plasmid obtained in the step 2), to give a clone, and
5) culturing the clone obtained in the step 4) on a leucine-free plate for 3 days to evaluate the binding ability of the substance to be tested (polypeptide) and pro-caspase-4 or caspase-4 in the yeast cell from the colony-forming ability on the leucine-free plate. In such a method, formation of a colony on a leucine-free plate is used as an index that indicates binding of pro-caspase-4 or caspase-4 with a substance to be tested (polypeptide).

The co-immunoprecipitation method is carried out by, for example,
1) obtaining a substance to be tested (polypeptide)-FLAG expression plasmid expressing a substance to be tested (polypeptide) having a FLAG sequence at the downstream of a signal sequence,
2) obtaining an HA-(pro)caspase-4 expression plasmid capable of expressing pro-caspse-4 or caspase-4 having an HA sequence at the N-terminal side,
3) co-transfecting transiently the substance to be tested (polypeptide)-FLAG expression plasmid obtained in the step 1) and the HA-(pro)caspase-4 expression plasmid obtained in the step 2) into a cell, to give a co-transfectant,
4) culturing the co-transfectant obtained in the step 3), to give a cultured cell,
5) obtaining a cell extract from the cultured cell obtained in the step 4),
6) adding an anti-FLAG antibody or an anti-HA antibody to the cell extract obtained in the step 5) to carry out co-immunoprecipitation, and
7) detecting the co-immunoprecipitation by Western blotting analysis. In such a method, in both cases of the anti-FLAG antibody and of the anti-HA antibody, precipitation of a complex of the substance to be tested (polypeptide)-pro-caspase-4 or the substance to be tested (polypeptide)-caspase-4 is used for evaluation as an index that indicates binding of pro-caspase-4 or caspase-4 with the substance to be tested (polypeptide).

The pull-down method is carried out by, for example,
1) contacting a fusion protein consisting of GST and pro-caspase-4 or caspase-4 (GST-(pro)caspase-4) with a substance to be tested (polypeptide),
2) collecting the fusion protein with glutathione beads, and
3) carrying out Western blotting analysis using an anti-substance to be tested (polypeptide) antibody, to detect a complex of GST-(pro)caspase-4 and the substance to be tested (polypeptide). In such a method, presence of a complex of GST-(pro)caspase-4 and the substance to be tested (polypeptide) is used for evaluation as an index that indicates binding of pro-caspase-4 or caspase-4 with the substance to be tested (polypeptide).

The plasmon resonance interaction analysis is carried out by
1) feeding a tip to which a substance to be tested (polypeptide) or pro-caspase-4 (or caspase-4) is immobilized with pro-caspase-4 (or caspase-4) or a substance to be tested at a constant flow rate correspondingly, and
2) detecting interaction by appropriate detection means (for example, a combination of optical detection (such as fluorescence value, fluorescence polarization value) and a mass spectrometer (matrix-assisted laser desorption ionization-time of flight mass spectrometer: MALDI-TOF MS, electrospray-ionization mass spectrometer: ESI-MS and the like). In such a method, presentation of sensorgram indicating formation of a complex consisting of pro-caspase-4 or caspase-4 and a substance to be tested is used for evaluation as an index that indicates binding of pro-caspase-4 or caspase-4 with the substance to be tested. Here, the plasmon resonance interaction analysis is advantageous regarding the applicability to various substances.

The pro-caspase-4 or the caspase-4 can be obtained by the method described in, for example, Kamada S., et al, Oncogene, 15, 285-290 (1997) (all teachings of which are incorporated herein by reference), Hana Bruchova, et al, Leuk. Lymphoma, 43, 1289-1295 (2002) (all teachings of which are incorporated herein by reference) and the like.

Here, in the evaluation method of the present invention, regarding the substance obtained in the step a), the configuration of the binding region of pro-caspase-4 or caspase-4 and the substance, and the three-dimensional coordinates of the amino acid residues present at the binding region and at its vicinity at a side-chain level can be identified and thereafter a compound adaptable to the identified configuration of the binding region and the three-dimensional coordinates of the amino acid residues present at the binding region and its vicinity can be designed.

Identification of the three-dimensional coordinates at a side-chain level can be obtained, for example, by analyzing the complex between pro-caspase-4 or caspase-4 and the substance obtained in the step a) on the conformation by 3D-NMR; crystallizing the complex, analyzing the resulting crystals on the conformation by X-ray crystal structure analysis, and analyzing by heavy-atom substitution method, isomorphic substitution method, multiwavelength anomalous diffraction method, molecular replacement method and the like. The design of the compound can be carried out by conventional computer-aided drug design.

Here, in the present specification, the “amino acid residues present at vicinity of the binding region” are not limited within the positional relation in a contiguous amino acid sequence at a primary structure level. The “amino acid residues present at vicinity of the binding region” refer to residues being within the region related to electrostatic interaction, hydrophobic interaction, van der Waals interaction, hydrogen bond and the like, meaning residues spatially located at the vicinity of a binding region in the conformation of the binding region.

Next, in the step b), the substance obtained in the step a) is brought into contact with caspase-4 in the presence of a substance causing endoplasmic reticulum stress or amyloid-β, to give a product.

In the step b), the cleaved product can be detected by electrophoresis, HPLC, MELD-MASS, immunostaining (Western blotting) and the like, and be dispensed if desired.

Subsequently, in the step c), a substance to be tested is prepared on the basis of the site of the product obtained in the step b) cleaved by caspase-4, as a derivative containing the cleavage site region of the product.

In the step c), the cleavage site of caspase-4 in the product obtained in the step b) can be analyzed by amino acid sequence analysis such as, for example, combination of degradation by conventional Edman degradation method, amino-terminal peptidase and the like and mass spectrometry, when the product is a peptide.

A derivative containing the circumference of the cleavage site of the product obtained in the step b) can be produced exhaustively by conventional method for synthesis and the like, for example, by combinatorial chemistry.

A cell expressing pro-caspase-4 includes, for example, SK-N-SH cell, HeLa cell, SY-SY cell, a transfected cell into which a nucleic acid encoding pro-caspase-4 is introduced, and the like. Host cell in the transfected cell includes HEK293 cell, HEK293T cell and the like. Here, each of the HEK293 cell and the HEK293T cell is a cell which does not express endogenous caspase-4. Therefore, transfected cell obtained by introducing a nucleic acid encoding pro-caspase-4 into the HEK293 cells or the HEK293T cells are advantageous since the behaviors mediated by caspase-4 can be detected more specifically.

In the present invention, the transfected cell into which a nucleic acid encoding the pro-caspase-4 is introduced includes:
A) a cell obtained by exogenously introducing a nucleic acid encoding pro-caspse-4 into a cell substantially lacking endogenous caspase-4 or a cell in which endogenous caspase-4 does not function substantially,
B) a cell prepared by exogenously introducing a nucleic acid encoding pro-caspase-4 into a cell carrying endogenous caspase-4,
C) a cell having a functional activity of pro-caspase-4, wherein the cell is a cell prepared by modifying or mutating a nucleic acid encoding endogenous caspase-4 by homogenous recombination and the like, and the like. In the cells of the A) to C), a nucleic acid encoding pro-caspase-4 or caspase-4 can be a nucleic acid modified so as to be able to be expressed as a fusion protein of pro-caspase-4 or caspase-4 with an appropriate fusion partner, for example, GFP, β-galactosidase, FLAG tag, poly-histidine tag, and the like.

When the nucleic acid encoding pro-caspase-4 is introduced into a cell, a expression vector obtained by operably linking the nucleic acid to a vector can be introduced into the cell. Here, the vector and the method for introducing a nucleic acid into a cell can be the same ones as described above.

A substance causing endoplasmic reticulum stress includes tunicamycin, thapsigargin, calcium ionophore A23187, 2-deoxyglucose and the like. When endoplasmic reticulum stress is caused by using the substance, the substance can be used, for example, so as to have a final concentration of 0.1 μM or more, preferably 1 μM or more, 100 μM or less, preferably, 10 μM or less. In addition, in the present invention, endoplasmic reticulum stress can be those caused by conditions such as glucose depletion, low-oxygen exposure (Brefeldin A, dithiothreitol and the like), and low-serum.

When amyloid-β is used, full-length amyloid-β can be used, and a partial peptide of the amyloid-β, for example, Aβ₂₅₋₃₅ and the like can be used. The amyloid-β can be used so as to have a final concentration of 0.1 μM or more, preferably 1 μM or more, 100 μM or less, preferably 10 μM or less. Here, in the present invention, a peptide having the function equivalent to the amyloid-β, for example, neurotoxin such as 6-hydroxydopamine can also be used.

In the present specification, “contacting a substance to be tested with a cell expressing pro-caspase-4” means a concept which encompasses culture or sustentation of a cell in the presence of a substance to be tested, direct or indirect introduction of a substance to be tested into a cell and the like.

Specifically, contact of a substance to be tested with a cell expressing pro-caspase-4 can be carried out, for example, by culturing or sustaining a cell expressing pro-caspase-4 in an appropriate culture medium or a buffer containing a substance to be tested, when the substance to be tested is a compound, a peptide or a peptide mimic. The contact can also be carried out by introducing a substance to be tested into a cell expressing pro-caspase-4 by microinjection and the like when the substance to be tested is a high-molecular compound such as a peptide.

The culture medium can be any culture medium suitable for culturing or sustaining the cell. The culture medium includes, for example, MEM, DMEM αMEM, F12 and the like. The culture medium can be used alone, or properly in combination. In addition, the culture medium can contain, if needed, various growth factors, serum, antibiotics and the like. A gas phase for culturing a cell in the presence of a substance to be tested can be appropriately set depending on the cell. The gas phase for culture includes, for example, 34° to 37° C., 0 to 5% by volume of CO₂ and the like. Specifically, for example, in the case of SK-N-SH cell, the conditions include culture in αMEM containing 10% by weight of fetal bovine serum at 37° C., 5% by volume of CO₂, and in the case of HeLa cell, the conditions include culture in DMEM containing 10% by weight of fetal bovine serum, at 37° C., 5% by volume of CO₂, culture under low oxygen, 0% by volume of 02, 5% by volume of CO₂, 95% by volume of N₂, and the like.

The above buffer includes Hanks' balanced salt solution, Krebs-Ringer solution, HEPES buffer, Tris buffer, phosphate buffer and the like. From the viewpoint of keeping physiological conditions of a cell, it is desirable that the buffer is pH7.0 or more, preferably pH7.2 or more, more preferably pH7.4 or more, and is pH8.5 or less, preferably pH7.8 or less, more preferably pH7.6 or less.

Time period for culture or sustentation can be appropriately set depending on the conditions for causing apoptosis (for example, endoplasmic reticulum stress, amyloid-β), the kind of the cell and the like. Specifically, in the case of tunicamycin, the time period can be for example, 6 to 72 hours, preferably 24 to 48 hours, and in the case of thapsigargin, the time period can be, for example, 1 to 72 hours, preferably 24 to 48 hours. In addition, in the case of amyloid-β, the time period can be, for example, 8 to 72 hours, preferably 8 to 24 hours.

In addition, when a substance to be tested is a peptide or a protein, a nucleic acid encoding the peptide or the protein can be operably linked to appropriate vector, and the expression vector thus obtained can be introduced into a cell. Here, the vector, the method for introducing a nucleic acid into a cell and the like can be the same ones as described above. When a substance to be tested is a nucleic acid or a nucleic acid analog, the nucleic acid or the nucleic acid analog can be directly introduced into a cell. The nucleic acid or the nucleic acid analog can be introduced into a cell via an expression vector obtained by linking the nucleic acid or the nucleic acid analog operably into an appropriate vector.

Introduction of a substance to be tested (nucleic acid and the like) into a cell can be carried out by the same means for introducing a gene as described above. Here, when a substance to be tested is a nucleic acid, the nucleic acid can be introduced into a cell by ligating the nucleic acid to an appropriate vector, or the nucleic acid can be directly introduced into a cell. The same vector as described above can be a vector to be used.

In addition, when the siRNA is introduced into a cell, a vector which can be used includes, but not particularly limited to, for example, trade name: pSilencer. 1.0-U6 (manufactured by Ambion) and the like.

In addition, in the evaluation method of the present invention, when a substance to be tested is siRNA, siRNA is synthesized in vitro, and the siRNA can be introduced into a cell by conventional technique.

Here, in the evaluation method of the present invention, when a substance to be tested is siRNA, the effect of the siRNA can be evaluated by using as a negative control, siRNA having the same base composition as that of the siRNA used.

When ribozyme is introduced into a cell, a vector used includes, for example, trade name: piGENEtRNA (manufactured by TAKARA BIO INC.) and the like.

The behavior mediated by the caspase-4 includes production of caspase-4 by cleavage of pro-caspase-4, condensation of chromatins, fragmentation of a chromosome, shrinkage of a cell, release of MTS, release of lactate dehydrogenase (LDH), generation of a product resulting from a substrate of caspase-4 and the like.

Generation of caspase-4 by cleavage of the pro-caspase-4 can be detected by, for example, preparing a cell extract from a cell, and carrying out Western blotting analysis, ELISA, and other immunological detection methods using an antibody which reacts with pro-caspase-4 but does not react with caspase-4 and an antibody which reacts with caspase-4 and reacts with pro-caspase-4. The condensation of chromatins can be detected by staining with a fluorescence dye which binds specifically to a DNA, and subsequently observing under a fluorescence microscope. Fragmentation of a chromosome can be detected as a ladder, by extracting a sample containing a chromosome and electrophoresing by a conventional electrophoresis. Shrinkage of a cell can be determined by equipment for determining a size distribution of cells, flow cytometry and the like. Release of MTS can be determined by a conventional measurement kit and the like. Specifically, release of MTS can be evaluated by, for example, measuring the amount of MTS released from a cell by measuring absorption at 490 nm by a spectrophotometer using an MTS solution (manufactured by Promega). In addition, behaviors of a cell can also be detected by TUNEL method.

Specifically, the evaluation method of the present invention can be carried out, for example, by a process (referred to as Process 1) including the steps of
(1) contacting a substance to be tested with a cell expressing pro-caspase-4,
(2) culturing the cell obtained in the step (1) in the presence of a substance causing endoplasmic reticulum stress or amyloid-β, and
(3) examining the cell after carrying out the step (2) on change specific for apoptosis, a process (referred to as Process 2) including the steps of
(A) culturing a cell expressing pro-caspase-4 with a substance to be tested in the presence of a substance causing endoplasmic reticulum stress or amyloid-β, and
(B) examining the cell after carrying out the step (A) on change specific for apoptosis. In the Process 1, behaviors mediated by caspase-4 in a cell are evaluated as change specific for apoptosis. In the Process 2, behaviors mediated by caspase-4 in a cell are evaluated over time as a change specific for apoptosis after step (A).

The change specific for apoptosis includes condensation of chromatins, fragmentation of a chromosome, shrinkage of a cell, release of MTS, release of LDH and the like.

Furthermore, in the evaluation method of the present invention, the presence or absence of changes in behaviors caused by the presence of a substance to be tested can be determined in the presence of a substrate of caspase-4.

According to the evaluation method of the present invention, I) a substance which inhibits expression of pro-caspase-4; a substance which inhibits binding of pro-caspase-4 and a substrate; a substance which inhibits cleavage of pro-caspase-4, and thus inhibits endoplasmic reticulum stress-induced and/or amyloid-β-induced apoptosis, II) a substance which acts on pro-caspase-4 to enhance production of caspase-4, and thus causing endoplasmic reticulum stress-induced and/or amyloid-β-induced apoptosis and the like can be evaluated.

The substance of the I) can be evaluated by using as an index the events so that pro-caspase-4 is cleaved in the absence of the substance, to thereby cause endoplasmic reticulum stress-induced and/or amyloid-β-induced apoptosis, but cleavage of pro-caspase-4 is substantially inhibited or reduced in the presence of the substance, whereby endoplasmic reticulum stress-induced and/or amyloid-β-induced apoptosis is not substantially caused or is reduced.

In addition, the substance of the II) can be evaluated according to the evaluation method of the present invention, for example, using as an index the events so that cleavage of pro-caspase-4 is increased in the presence of the substance as compared with that in the absence of the substance, and endoplasmic reticulum stress-induced and/or amyloid-β-induced apoptosis is increased. The substance of the II) is also useful for, for example, treatment of cancer (chronic myelogenous leukemia and the like).

Here, in the evaluation method of the present invention, whether or not cleavage of pro-caspase-4, apoptosis mediated by caspase-4, effects of a substance to be tested and the like are specific for endoplasmic reticulum stress-induced and/or amyloid-β-induced apoptosis can be evaluated by using as a control a behavior due to a substance causing endoplasmic reticulum stress, amyloid-β or a substance other than neurotoxin equivalent to the amyloid-β, for example, etoposide, staurosporine and the like, or a behavior under the conditions where endoplasmic reticulum stress is not caused, for example, UV irradiation and the like.

In addition, the evaluation method of the present invention includes an embodiment wherein inhibition or enhancement of action of caspase-4 in signal transduction of apoptosis in a cell is evaluated as an index. In this case, action of caspase-4 includes cysteine protease activity and the like.

Other aspect of the present invention is a kit for evaluating a substance capable of affecting endoplasmic reticulum stress-induced apoptosis and/or amyloid-β-induced apoptosis, for carrying out the evaluation method of the present invention, which includes 1) a cell expressing pro-caspase-4, 2) a substance causing endoplasmic reticulum stress or amyloid-β, 3) a reagent suitable for contacting a substance to be tested with the cell.

Since the kit for evaluation of the present invention contains the above items 1) to 3), a substance capable of affecting endoplasmic reticulum stress-induced apoptosis and/or amyloid-β-induced apoptosis can be evaluated rapidly, easily, well-repeatably, and accurately, according to the evaluation method of the present invention. Since the kit for evaluation of the present invention contains the above items 1) to 3), the evaluation method of the present invention can also be carried out more reflecting conditions in a living body.

In the kit for evaluation of the present invention, the cell expressing pro-caspase-4 of the above item 1) can be stored in a culture medium suitable for sustaining the cell. The culture medium includes MEM, DMEM, αMEM, F12 and the like.

In the kit for evaluation of the present invention, the substance causing endoplasmic reticulum stress or amyloid-β of the above item 2) can be in the form of either powder, liquid or the like, and can be stored in a solution suitable for storing the substance or amyloid-β, for example, in a buffer. The buffer includes phosphate buffered saline, Tris buffer, HEPES buffer and the like. From the viewpoints of keeping physiological conditions of a cell and keeping biological activities of the substance or amyloid-β, it is desirable that such a solution has a pH of 7.0 or more, preferably a pH of 7.2 or more, more preferably a pH of 7.4 or more, and has a pH of 8.5 or less, preferably a pH of 7.8 or less, more preferably a pH of 7.6 or less.

The reagent of the above item 3) suitable for contacting the substance to be tested with the cell includes the culture medium, the buffer and the like.

Here, the kit for evaluation of the present invention can also be provided as a group of reagents dispensed in advance so as to have the amount of cell with a dilution ratio suitable for carrying out the evaluation method of the present invention or the amount of the substance causing endoplasmic reticulum stress or amyloid-β with a dilution ratio suitable for carrying out the evaluation method of the present invention.

The kit for evaluation of the present invention can further contain a vessel suitable for culturing, a reagent for preparing a cell extract and the like.

Another aspect of the present invention relates to a pharmaceutical composition, which contains as an active ingredient, a substance evaluated by the evaluation method of the present invention, wherein the substance is a substance capable of affecting endoplasmic reticulum stress-induced apoptosis and/or amyloid-β-induced apoptosis. Since the pharmaceutical composition of the present invention contains a substance capable of affecting endoplasmic reticulum stress-induced apoptosis and/or amyloid-β-induced apoptosis as an active ingredient, a disease or progress thereof caused by endoplasmic reticulum stress-induced and/or amyloid-β-induced apoptosis can be inhibited according to the pharmaceutical composition of the present invention. Therefore, when action of caspase-4 plays a role in onset or progress in signal transduction of apoptosis, the pharmaceutical composition of the present invention is especially useful.

The “disease or progress thereof caused by endoplasmic reticulum stress-induced and/or amyloid-β-induced apoptosis” includes epilepsy; neurodegenerative disease, for example, Alzheimer's disease, Parkinson's disease, amyotrophic lateral sclerosis, frontotemporal dementia, corticobasal degeneration, Huntington disease and the like; ischemic disease, for example, cerebral ischemia, myocardial infarction, stroke and the like; type I diabetes and the like.

Specifically, the pharmaceutical composition of the present invention contains the substance of the above items I), II) and the like obtained by the evaluation method of the present invention as an active ingredient.

When the active ingredient of the pharmaceutical composition of the present invention is the substance of the above item I), such a pharmaceutical composition can be applied to epilepsy, Alzheimer's disease, Parkinson's disease, amyotrophic lateral sclerosis, cerebral ischemia, frontotemporal dementia, corticobasal degeneration, Huntington disease and the like.

The substance of the above I) includes a substance obtained from the above substance to be tested using the evaluation method of the present invention, for example, an siRNA specific for pro-caspase-4 or caspase-4, an antisense nucleic acid to a nucleic acid encoding pro-caspase-4 or caspase-4, a ribozyme for a nucleic acid encoding pro-caspase-4 or caspase-4 and the like.

The siRNA includes specifically, but not particularly limited to, a double-stranded nucleic acid consisting of a nucleic acid consisting of the sequence shown in SEQ ID NO: 3 and a nucleic acid consisting of the sequence shown in SEQ ID NO: 4; a double-stranded nucleic acid consisting of a nucleic acid consisting of the sequence shown in SEQ ID NO: 5 and a nucleic acid consisting of the sequence shown in SEQ ID NO: 6, and the like.

Also, when the active ingredient of the pharmaceutical composition of the present invention is the substance of the above item II), the pharmaceutical composition of the present invention can be applied to, for example, viral infection, cancer and the like. In this case, it is desirable that the pharmaceutical composition of the present invention preferably further contains a component suitable for delivering the substance of the above item II) to a site to be applied, for example, viral-infection site, cancer tissue and the like. The component suitable for delivering the substance of the above item II) to a site to be applied includes, for example, a ligand, a receptor, an antibody and the like to a factor expressing specifically for the site to be applied.

The active ingredient of the pharmaceutical composition of the present invention can be made to be carried by a pharmaceutically acceptable carrier suitable for introducing into an individual, an organ, a local site, a tissue and the like.

The pharmaceutical composition of the present invention can further contain other auxiliary agent depending on the disease to be applied, or the individual, the organ, the local site or the tissue to be applied. Specifically, when the active ingredient is a nucleic acid, a nucleic acid analog, a peptide or a peptide mimic, a pharmaceutically acceptable auxiliary agent, excipient, binding agent, stabilizer, buffer, solubilizing agent, isotonic agent and the like exhibiting properties to suppress degradation of the nucleic acid, nucleic acid analog, peptide or peptide mimic can be included, by the process in which the active ingredient reaches a site where the effect of the active ingredient is to be expressed.

The active ingredient content in the pharmaceutical composition of the present invention can be a therapeutically effective amount, and can be appropriately set depending on the kind of a substance as an active ingredient, an individual to be applied, weight of the individual, age of the individual and the like. Specifically, when the active ingredient is a nucleic acid, it is desirable that the active ingredient content is about 0.001 mg/kg or more, preferably about 0.005 mg/kg or more, more preferably 0.01 mg/kg or more per day, and is about 2.0 mg/kg or less, preferably about 1.0 mg/kg or less, more preferably about 0.5 mg/kg or less per day, from the viewpoint of activity, solubility, absorbability, biological half-life and the like. In addition, when the active ingredient is a peptide or a peptide mimic, it is desirable that the active ingredient content is about 0.001 mg/kg or more, preferably about 0.005 mg/kg or more, more preferably about 0.01 mg/kg or more per day, and is about 2.0 mg/kg or less, preferably about 1.0 mg/kg or less, more preferably about 0.5 mg/kg or less per day, from the viewpoint of activity, solubility, absorbability, biological half-life and the like. Furthermore, when the active ingredient is a compound other than the nucleic acid and the peptide, it is desirable that the active ingredient content is about 0.001 mg/kg or more, preferably about 0.005 mg/kg or more, more preferably 0.01 mg/kg or more per day, and is about 2.0 mg/kg or less, preferably about 1.0 mg/kg or less, more preferably about 0.5 mg/kg or less per day, from the viewpoint of activity, solubility, absorbability, biological half-life and the like.

Administration forms and dose of the pharmaceutical composition of the present invention can be appropriately selected depending on the kind of the active ingredient, disease to be applied, an individual, an organ, a local site, a tissue to be applied, age, weight and the like of an individual to be applied. The administration forms include local administration; hypodermic injection; intramuscular injection; intravenous injection; oral administration via a tablet, a capsule, a granule, syrup and the like.

In addition, when the active ingredient of the pharmaceutical composition of the present invention is a nucleic acid component, the pharmaceutical composition can be directly introduced into a body as a virus vector or a liposome carrying a nucleic acid of the active ingredient, and also can be introduced extarcorporeally into a certain kind of a cell of the individual to be applied and the like, thereafter transferring back the resulting cell into the body, whereby the pharmaceutical composition is introduced into the body. Specifically, for example, in the case of Alzheimer's disease, the pharmaceutical composition of the present invention can be administered by, for example, injecting locally the pharmaceutical composition of the present invention into a specific site in a brain or introducing the pharmaceutical composition of the present invention into a cell such as an astrocyte, thereafter transplanting the resulting cell into a brain.

Evaluation of the effect of the pharmaceutical composition of the present invention can be carried out by, for example, using amelioration or cure of a symptom of a disease, lowering or disappearance of pathological properties and the like as an index, as compared with a model animal against the above disease for administration and non-administration of the pharmaceutical composition of the present invention (for example, a model with intraventricular administration of β-myloid, a model for middle cerebral artery occulation, a model for 6-hydroxydopamine Parkinson), a knock-in animal into which a nucleic acid encoding human caspase-4 was introduced, and the like with a non-administered animal.

Still another aspect of the present invention relates to a method for treating or preventing a disease caused by the endoplasmic reticulum stress-induced apoptosis and/or amyloid-β-induced apoptosis, which comprises administering a substance evaluated by the evaluation method of the present invention, which is a substance capable of affecting endoplasmic reticulum stress-induced apoptosis and/or amyloid-β-induced apoptosis to an individual suffering from the disease. In the method for treatment or prevention of the present invention, since the “substance evaluated by the evaluation method of the present invention, which is a substance capable of affecting endoplasmic reticulum stress-induced apoptosis and/or amyloid-β-induced apoptosis” is used, according to such a method for treatment or prevention of the present invention, treatment or prevention of endoplasmic reticulum stress-induced apoptosis and/or amyloid-β-induced apoptosis, especially on the basis of caspase-4 can be achieved.

The dose of the “substance evaluated by the evaluation method of the present invention, which is a substance capable of affecting endoplasmic reticulum stress-induced apoptosis and/or amyloid-β-induced apoptosis” can be a therapeutically effective dose, and can be appropriately set as in the case with the above pharmaceutical composition. Administration form of the substance is also the same as in the above pharmaceutical composition.

The effect of treatment or prevention by the method for treatment or prevention of the present invention can be evaluated by using decrease in events characteristic in a disease of an individual to be applied, lowering of learning ability and the like as an index. For example, in the case of Alzheimer's disease, when decrease in development of degenerated neurocytes, retardation of learning ability, lowering (retention) of grade of dementia and the like are found in the individual by applying the method for treatment or prevention of the present invention, it is used as an index which indicates that the effect of the treatment or prevention is exhibited. In addition, in the case of cerebral ischemia, when diminution of ischemic site, amelioration of paralysis and the like are found, it is used as an index which indicates that the effect is exhibited.

Furthermore, another aspect of the present invention relates to a use of a substance which is evaluated by the evaluation method of the present invention which is a substance capable of affecting endoplasmic reticulum stress-induced apoptosis and/or amyloid-β-induced apoptosis, for producing a medicinal for treating or preventing the disease caused by endoplasmic reticulum stress-induced and/or amyloid-β-induced apoptosis by administering to an individual suffering from the disease.

In addition, other aspect of the present invention relates to a method for diagnosis of the disease caused by endoplasmic reticulum stress-induced and/or amyloid-β-induced apoptosis on the basis of increase or decrease in caspase-4 resulting from cleavage of pro-caspase-4 or increase or decrease in a reaction product generated from an in vivo substrate of caspase-4. Here, such a method for diagnosis can be applied to a method for detecting a sample from an individual suffering from the disease caused by endoplasmic reticulum stress-induced and/or amyloid-β-induced apoptosis.

The method for diagnosis of the present invention can be carried out by, for example,
1. detecting increase or decrease of caspase-4 resulting from cleavage of pro-caspase-4 in a sample from a tissue or an organ in which a disease can develop, using an antibody or a fragment thereof which does not react with pro-caspase-4 but reacts with caspase-4 and/or an antibody or a fragment thereof which reacts with pro-caspase-4 but does not react with caspase-4;
2. detecting increase or decrease of a reaction product generated from an in vivo substrate by the effect of caspase-4 in a sample from a tissue or an organ in which a disease can develop, using an antibody which reacts with a reaction product generated from an in vivo substrate by the effect of caspase-4;
3. detecting increase or decrease of a nucleic acid (such as, for example, mRNA) encoding a reaction product generated from an in vivo substrate of caspase-4 in a nucleic acid-containing sample extracted from a sample from a tissue or an organ in which a disease can develop, using a nucleic acid which specifically binds to a nucleic acid encoding a reaction product generated from an in vivo substrate by the effect of caspase-4; and the like. Here, significant change in the amount of caspase-4 resulting from cleavage of pro-caspase-4, a nucleic acid encoding the caspase-4, a reaction product generated from an in vivo substrate of caspase-4, a nucleic acid encoding the reaction product and the like is an index of possibility that the individual to be tested is suffering from a disease caused by endoplasmic reticulum stress-induced and/or amyloid-β-induced apoptosis.

The sample includes, for example, an exudate, blood, tissue biopsy, cerebrospinal fluid and the like from an individual, specifically from a patient with dementia, a patient with Parkinson's disease and the like.

The antibody can be a polyclonal antibody, or can be a monoclonal antibody. Furthermore, in the present invention, the antibody can be an antibody or a derivative thereof modified according to a known technique, for example, a humanized antibody, a chimeric antibody, a single-chain antibody and the like. In addition, the fragment of the antibody can be a product obtained by digesting the antibody with peptidase, for example, papain, pepsin and the like. The fragment of the antibody includes, for example, a Fab fragment obtained by digesting a monoclonal antibody with papain, F(ab′)₂ fragment obtained by digesting a monoclonal antibody with pepsin and the like. The antibody can be easily produced by the method described in John E. Coligan Ed., Current Protocols in Immunology, John Wiley & Sons, Inc., 1992 (all teachings of which are incorporated herein by reference). Here, pro-caspase-4, caspase-4, a peptide consisting of a region which exists in pro-caspase-4 but does not exist in caspase-4, a reaction product generated from an in vivo substrate by the effect of caspase-4 and the like can be used in immunization of an animal upon producing an antibody. In addition, the antibody can be genetically engineered. Furthermore, a fragment of an antibody can also be obtained by processing the resulting antibody with peptidase and the like after purification. The antibody or the fragment thereof can be labeled with an enzyme, a fluorescent substance, a radioactive substance and the like.

The antibody or the fragment thereof which does not react with pro-caspase-4 but reacts with caspase-4 and the antibody or the fragment thereof which reacts with pro-caspase-4 but does not react with caspase-4 are evaluated by, for example, carrying out ELISA, Ouchterlony method and the like using pro-caspase-4, caspase-4, a peptide which exists in pro-caspase-4 but not in caspase-4 and the like, to examine the cross-reactivity for pro-caspase-4 and caspase-4.

The antibody which reacts with a reaction product generated from an in vivo substrate by the action of caspase-4 is also evaluated by carrying out ELISA, Ouchterlony method and the like using the reaction product and the like, to examine reactivity against the reaction product.

In the method for diagnosis of the present invention, in a case where an antibody or a fragment of an antibody is used, the amount of caspase-4 or of the reaction product can be detected by a method such as, for example, Western blotting, antibody column method, ELISA, immunoprecipitation.

The nucleic acid can be designed on the basis of a sequence having low sequence identity to a known sequence in a sequence of a nucleic acid encoding a reaction product generated from an in vivo substrate by the effect of caspase-4 and the like, for example, on the basis of a sequence appropriately aligned to a reference sequence under the similar parameter as described above on the basis of BLAST algorism, wherein the sequence identity obtained is preferably 15% or less, more preferably 10% or less, even more preferably 5% or less, even further more preferably 0%. Here, the nucleic acid used in the method for diagnosis of the present invention can be a primer pair which specifically amplifies a nucleic acid or a portion thereof encoding a reaction product (i.e., a primer pair which can specifically amplify a sequence having low sequence identity with a known sequence in a sequence of a nucleic acid encoding a reaction product generated from an in vivo substrate by the action of caspase-4) and the like.

Here, length of the primer is not particularly limited, but it is desirable that the primer is preferably at least 10 continuous nucleotides in length, more preferably 10 to 50 continuous nucleotides in length, even more preferably 15 to 25 continuous nucleotides in length.

In the method for diagnosis of the present invention, in the case where a nucleic acid is used, a nucleic acid encoding caspase-4 or a nucleic acid encoding the reaction product can be quantitatively detected by Southern blotting analysis, Northern blotting analysis, hybridization by DNA array, PCR, RT-PCR and the like.

In addition, according to the present invention, a kit for diagnosis of the disease caused by endoplasmic reticulum stress-induced and/or amyloid-β-induced apoptosis, which contains at least one kind selected from the group consisting of the antibody or the fragment thereof which does not react with pro-caspase-4 but reacts with caspase-4, the antibody or the fragment thereof which reacts with pro-caspase-4 but not with caspase-4, the antibody which reacts with a reaction product generated from an in vivo substrate by the action of caspase-4, a nucleic acid which specifically binds to a nucleic acid encoding pro-caspase-4 but not binds to a nucleic acid encoding caspase-4, a primer pair which specifically amplifies a region which is different between pro-caspase-4 and caspase-4, a nucleic acid which specifically binds to a nucleic acid encoding a reaction product generated from an in vivo substrate by the action of caspase-4, and a primer pair which specifically amplifies a nucleic acid or a fragment thereof encoding a reaction product.

According to the evaluation method of the present invention, there is exhibited an excellent effect that screening and/or validation of a substance which specifically affects endoplasmic reticulum stress-induced and/or amyloid-β-induced apoptosis. In addition, according to the evaluation method of the present invention, there is exhibited an excellent effect that screening and/or validation of a substance capable of inhibiting a disease or progress thereof caused in association with endoplasmic reticulum stress-induced and/or amyloid-β-induced apoptosis can be carried out and that means for treating or preventing the disease can be developed. Furthermore, according to the kit for evaluation of the present invention, there is exhibited an excellent effect that the above method can be carried out rapidly, simply, well-repeatably, and accurately, with reflecting the conditions in a living body. In addition, according to the pharmaceutical composition of the present invention, there is exhibited an excellent effect that a disease or progress thereof caused by endoplasmic reticulum stress-induced and/or amyloid-β-induced apoptosis can be inhibited.

According to the present invention, treatment or prevention of a disease caused in association with endoplasmic reticulum stress-induced apoptosis and/or amyloid-β-induced apoptosis such as neurodegenerative disease, ischemic disease is possible. According to the present invention, screening or pharmacological evaluation of means for treating or preventing the disease, a drug which is effective for the disease and the like is also possible.

The present invention will be explained in more detail hereinbelow by Examples, but the present invention is not limited by the Examples.

### EXAMPLE 1

Factors causing endoplasmic reticulum stress-induced apoptosis were screened in various kinds of human DNA libraries. As a result, a nucleic acid encoding a candidate factor in human large intestine cDNA library (manufactured by Stratagene) was found. It was also found that the nucleic acid has a sequence identical to a partial sequence of mouse caspse-12 gene.

Thereafter, the nucleic acid obtained was analyzed on the sequence. As a result, the nucleic acid was identified as a nucleic acid encoding human caspase-4. Namely, the presence of a factor exhibiting unexpectedly the same action as mouse caspase-12 was found, although the factor was found in cDNA library of a large intestine having lower amount of expression of the caspase-4 than that of caspase-5.

Localization of the human caspase-4 in a cell was then examined.

Human neuroblastoma SK-N-SH cells were cultured in α-MEM (manufactured by Invitrogen) containing 10% by weight of fetal bovine serum, at 37° C. under 5% by volume of CO₂ for 20 minutes with or without trade name: Mitotracker (manufactured by Molecular Probe) which can stain a mitochondrion. In addition, human carcinoma HeLa cells were cultured in DMEM (Invitrogen) containing 10% by weight of fetal bovine serum, at 37° C. under 5% by volume of CO₂ for 20 minutes, in the same manner.

Cultured SK-N-SH cells and HeLa cells were fixed by sustaining in 0.1 M phosphate buffer containing 4% by weight of paraformaldehyde for 2 hours at 4° C.

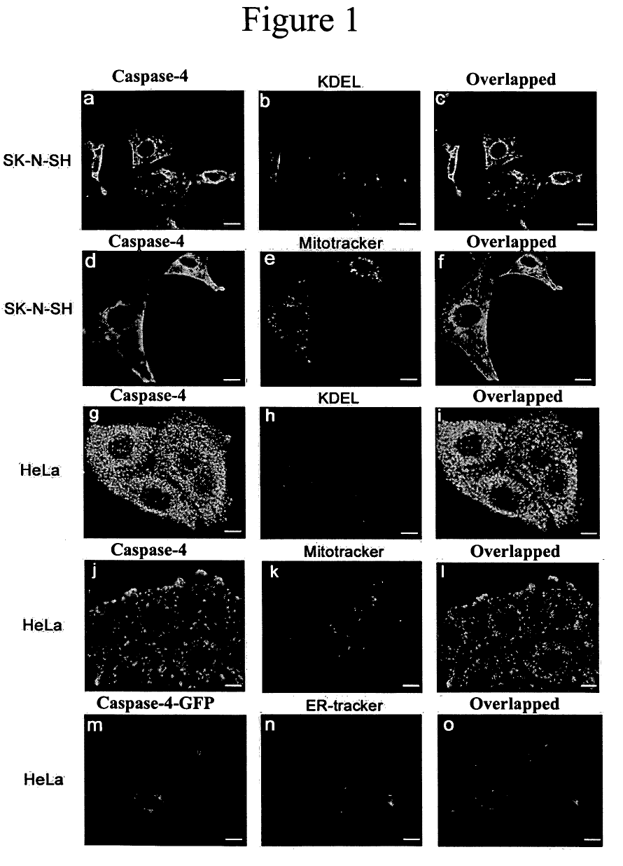

Cells after fixation were then incubated with anti-human caspase-4 monoclonal antibody (trade name: 4B9, manufactured by MEDICAL & BIOLOGICAL LABORATORIES CO., LTD) with or without anti-human KDEL monoclonal antibody (10C3, manufactured by Stressgen) which can detect both GRP78 and GRP94 which are endoplasmic reticulum markers. The cells were incubated with FITC-conjugated anti-goat IgG antibodies (manufactured by Jackson) or Alexa588-conjugated anti-mouse IgG antibodies (manufactured by Molecular Probes), respectively. Thereafter, stained cells were observed under a confocal microscope (trade name: LSM510, manufactured by Carl Zeiss), to observe cellular localization of endogenous caspase-4 in cells. The results are shown in Panels “a” to “l” of FIG. 1.

In addition, HeLa cell was transfected with a nucleic acid encoding caspase-4, which was subcloned into a pcDNA 3.1-GFP-tagged plasmid (manufactured by Invitrogen). After 24 hours, transfected cells were incubated with trade name: ER-tracker for 30 minutes, and thereafter observed under a fluorescence microscope. The results are shown in Panels “m” to “o” of FIG. 1.

As a result, as shown in Panels “a” to “c” of FIG. 1, it is found that immunoreactivity to caspase-4 was strictly co-localized with that of endoplasmic reticulum markers such as GRP78 and GRP94. However, as shown in Panels “d” to “f” of FIG. 1, it is found that the results are not consistent with the results of the staining of trade name: Mitotracker. The similar results were also obtained using HeLa cells as shown in Panels “g” to “l” of FIG. 1.

On the other hand, as shown in Panels “m” to “o” of FIG. 1, it is found that when caspase-4 fused with GFP was overexpressed in HeLa cells to observe the cellular localization in live cells, most of the fluorescent signals from caspase-4/GFP fusion protein overlapped with those from trade name: ER-tracker. These results show that caspase-4 was preferentially localized in the endoplasmic reticulum rather than in the mitochondria in both SK-N-SH cells and HeLa cells.

### EXAMPLE 2

SK-N-SH cells were treated with various kinds of apoptosis inducers such as tunicamycin (manufactured by SIGMA), thapsigargin (manufactured by SIGMA), etoposide (manufactured by SIGMA), staurosporine (manufactured by SIGMA), UV and the like, to evaluate change in caspase-4. Specifically, SK-N-SH cells were treated either with exposure to an endoplasmic reticulum stress inducer, tunicamycin (1 μg/ml) or thapsigargin (0.5 μM), exposure to a non-endoplasmic reticulum stress inducer, etoposide (100 μM) or staurosporine (0.1 μM), or irradiated with 150 J/m2 UV, in α-MEM (manufactured by Invitrogen) containing 10% by weight of fetal bovine serum, at 37° C. under 5% by volume of CO₂. Here, the exposures to tunicamycin, thapsigargin and etoposide were 16 or 24 hours, exposure to staurosporine was 8 hours, and UV irradiation was 8 hours.

Thereafter, the cells obtained were washed with phosphate buffered saline, and then harvested. The resulting cells were lysed in TNE buffer (composition: 10 mM Tris-HCl, pH 7.8, 1 mM EDTA, 150 mM NaCl, 1 mM phenylmethylsulphonyl fluoride) containing 0.5% by weight of NP-40 (trade name: Nonidet P-40), to give a cell extract. Equal amount of a cell extract (corresponding to 15 μg of protein) was subjected to 12% by weight SDS-PAGE. After the SDS-PAGE, proteins on the gel were transferred to a PVDF membrane (manufactured by Millipore). The membrane was blocked with 5% by weight of bovine serum albumin (BSA). The resulting membrane was incubated with a primary antibody (anti-caspase-4 antibody, anti-caspase-3 antibody, anti-caspase-7 antibody or anti-β-actin antibody), and thereafter incubated with a horseradish peroxidase (HRP)-conjugated secondary antibody. Thereafter, caspase-4, caspase-3, caspase-7 or β-actin was detected with trade name: ECL detection system (manufactured by Amersham-Pharmacia).

In the same manner as described above, cellular morphological changes of SK-N-SH cells treated with various apoptosis inducers were evaluated by phase contrast microscopy. SK-N-SH cells treated in the same manner were stained with 10 μM Hoechst 33342. Nuclear morphological changes of stained cells were evaluated by fluorescence microscopy. Cell death was detected by using as an index cellular morphological changes and nuclear morphological changes. At least 500 cells were counted. The data was expressed as the mean ±SEM from three independent experiments and P values were calculated by Student's t-test.

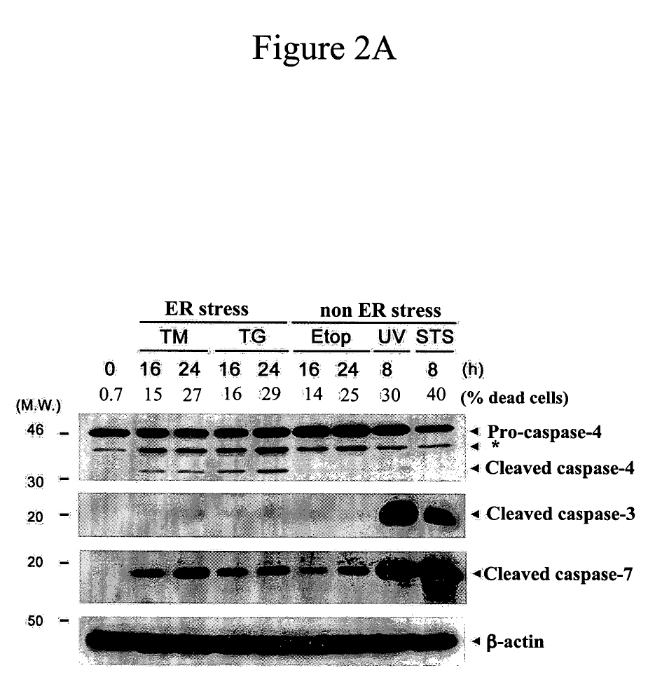

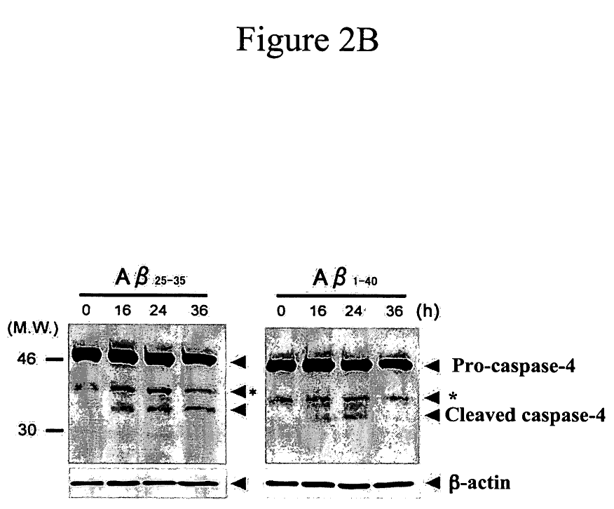

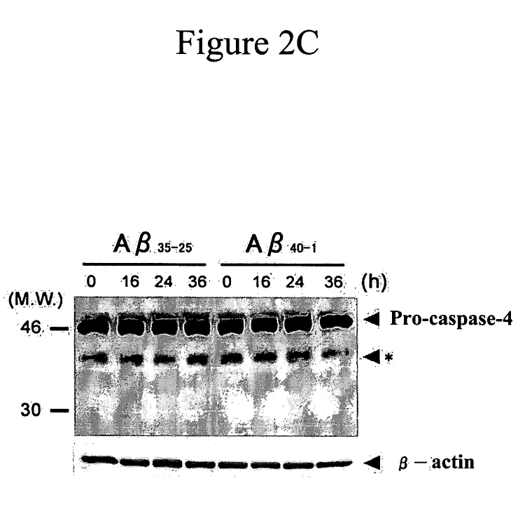

Furthermore, cells treated with in the same manner as described above were incubated with MTS solution (manufactured by Promega) for 1 hour at 37° C. The amount of MTS released from the viable cells was then quantified by measuring the absorbance at 490 nm using a spectrophotometer. Results are expressed as a ratio (%) of dead cells after treatments to dead cells in control as above. The results are shown in FIG. 2A.

As a result, as shown in FIG. 2A, it is found that treatment of SK-N-SH cells with endoplasmic reticulum stress inducers, tunicamycin or thapsigargin induced the cleavage of pro-caspase-4.

In contrast, as shown in FIG. 2A, although the extent of cell death by non-endoplasmic reticulum stress inducers such as etoposide, staurosporine, and UV was similar to that by tunicamycin and thapsigargin, it is found that pro-caspase-4 is not cleaved when cells were exposed to the non-endoplasmic reticulum stress inducers.

In addition, as shown in FIG. 2A, under the same conditions as in the pro-caspase-4, cleavage of caspase-7 was observed regardless of apoptotic stimulations.

These results suggest that caspase-4 is specifically activated by apoptotic stimuli inducing endoplasmic reticulum stress, but the caspase-4 is not specifically activated by other stimuli that do not cause endoplasmic reticulum stress.

The cleavage of caspase-4 in SK-N-SH cells after treatment with amyloid-β (Aβ) (manufactured by SIGMA-ALDRICH Corp.), a partial peptide thereof or a derivative peptide thereof was then examined.

SK-N-SH cells were incubated in α-MEM (manufactured by Invitrogen) containing 10% by weight of fetal bovine serum, at 37° C. under 5% by volume of CO₂, with 25 μM Aβ₂₅₋₃₅, 5 μM Aβ₁₋₄₀, 25 μM Aβ₃₅₋₂₅, or 5 M Aβ₄₀₋₁. Caspase-4, caspase-7 or β-actin in the cell extract was then detected in the same manner as described above. The results are shown in FIGS. 2B and 2C.

As a result, as shown in FIG. 2B, when the cells were incubated with 25 μM amyloid-β (Aβ)₂₅₋₃₅ or 5 μM Aβ₁₋₄₀, cleavage of caspase-4 was observed. In contrast, as shown in FIG. 2C, when the cells were incubated with the reverse peptides (Aβ₃₅₋₂₅ or Aβ₄₀₋₁, respectively), the cleavage of caspase-4 was not observed. These results suggest that caspase-4 is activated by neurotoxic Aμ treatment similar to endoplasmic reticulum stress-induced apoptosis.

### EXAMPLE 3

SK-N-SH cells were stably transfected with pCAGGS-hBcl-2 (Iwahashi, H. et al., Nature, 390, 413-417 (1997), all teachings of which are incorporated herein by reference) by a conventional method. The resulting cells were then cultured in αMEM (manufactured by Invitrogen) containing 10% by weight of fetal bovine serum, at 37° C. under 5% by volume of CO₂ to overexpress Bcl-2. Similarly, HeLa cells were stably transfected with pCAGGS-hBcl-2. The resulting cells were then cultured in DMEM (manufactured by Invitrogen) containing 10% by weight of fetal bovine serum, at 37° C. under 5% by volume of CO₂ for 48 hours to overexpress Bcl-2. Here, SK-N-SH cells and HeLa cells were stably transfected with trade name: pCAGGS (Hitoshi Niwa et al., Gene, 108, 193-200 (1991), all teachings of which are incorporated herein by reference) as controls, in the same manner as described above, respectively.

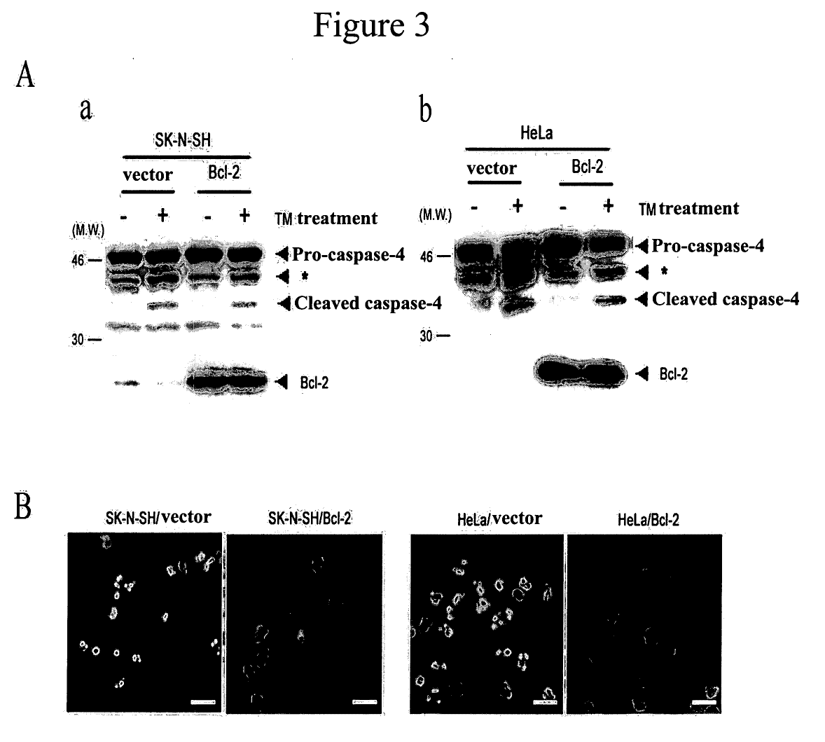

Each of the transfectants obtained was incubated with or without tunicamycin (1 μg/ml) for 16 hours. Cell extract was then prepared from the resulting cells in the same manner as in the above Example 2, and subjected to 12% by weight SDS-PAGE. After SDS-PAGE, proteins on the gel were transferred to a PVDF membrane (manufactured by Millipore). The membrane was blocked with 5% by weight of bovine serum albumin (BSA). The resulting membrane was then incubated with a primary antibody (anti-caspase-4 antibody or anti-Bcl-2 antibody (Cat. No. 100, manufactured by Pharmingen)), and thereafter incubated with an HRP-conjugated secondary antibody. Caspase-4 or Bcl-2 was then detected with trade name: ECL detection system (manufactured by Amersham-Pharmacia). The results are shown in FIG. 3A.

In addition, each of the transfectants was treated with tunicamycin (1 μg/ml) for 30 hours, stained with Hoechst33342, and observed by fluorescence microscopy. The results are shown in FIG. 3B.

As a result, as shown in FIG. 3B, it is found that apoptotic nuclear morphological changes were induced by treatment of the cell into which a vector was introduced (SK-N-SH cell and HeLa cell) with tunicamycin for 30 hours, but such changes were completely suppressed by overexpression of Bcl-2. Therefore, it is indicated that the downstream apoptotic signaling pathway was not functioning in cells with overexpression of these anti-apoptotic proteins.

However, as shown in FIG. 3A, cleavage of caspase-4 sixteen hours after tunicamycin treatment was only slightly affected by overexpression of Bcl-2. These results suggest that caspase-4 is largely activated before the activation of effector caspases during endoplasmic reticulum stress-induced cell death.

Here, with pCAGGS-hBcl-XL (Tagami, S. et al., Oncogene, 19, 5736-5746 (2000), all teachings of which are incorporated herein by reference), effects of overexpression of BCI-XL on cleavage of caspase-4 and nuclear morphological changes were examined in the same manner as in the case with the pCAGGS-hBcl-2.

Therefore, it is suggested that cleavage of caspase-4 was not due to downstream mitochondrial pathway activated by other caspases.

### EXAMPLE 4

It was determined whether or not caspase-4 is required for endoplasmic reticulum stress-induced cell death, using siRNA against caspase-4 or siRNA against GFP as a control. Annealed double-stranded siRNAs (manufactured by Dharmacon) were used to reduce expression of caspase-4 gene or GFP gene. The annealed double-stranded siRNAs are as follows: for caspase-4, an siRNA-a consisting of 5′-AAGUGGCCUCUUCACAGUCAUdTdT-3′ (SEQ ID NO: 3 (sense)) and 5′-AAAUGACUGUGAAGAGGCCACdTdT-3′ (SEQ ID NO: 4 (anti-sense)); and an siRNA-b consisting of 5′-AAGAUUUCCUCACUGGUGUUUdTdT-3′ (SEQ ID NO: 5 (sense)) 5′-AAAAACACCAGTGAGGAAATCdTdT-3′ (SEQ ID NO: 6 (anti-sense)); for green fluorescent protein (GFP), an siRNA consisting of 5′-PGGCUACGUCCAGGAGCGCACC-3′ (SEQ ID NO: 7 (sense)) and 5′-PUGCGCUCCUGGACGUAGCCUU-3′ (SEQ ID NO: 8 (anti-sense)).

Here, the sequences shown in SEQ ID NOs: 3 to 8 were those sequences which were not remarkably homologous to genes other than caspase-4 gene or GFP gene according to BLAST search (NCBI, updated data dated May 19, 2004).

SK-N-SH cell was transfected at 50% of confluence with 1.0 μg of the siRNA in 24-well plastic plates (manufactured by Nunc) containing αMEM (manufactured by Invitrogen) containing 10% by weight of fetal bovine serum, at 37° C. under 5% by volume of CO₂, using trade name: Oligofectamine (manufactured by Invitrogen) according to the manufacturer's protocol. Transfected SK-N-SH cells were incubated at 37° C. for 60 hours without changing the medium.

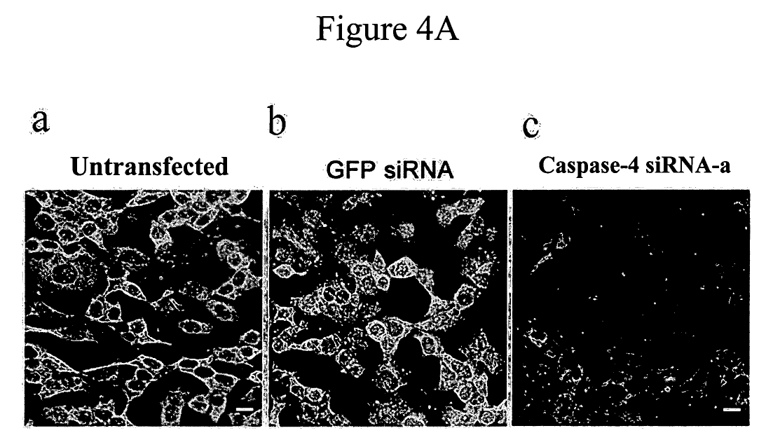

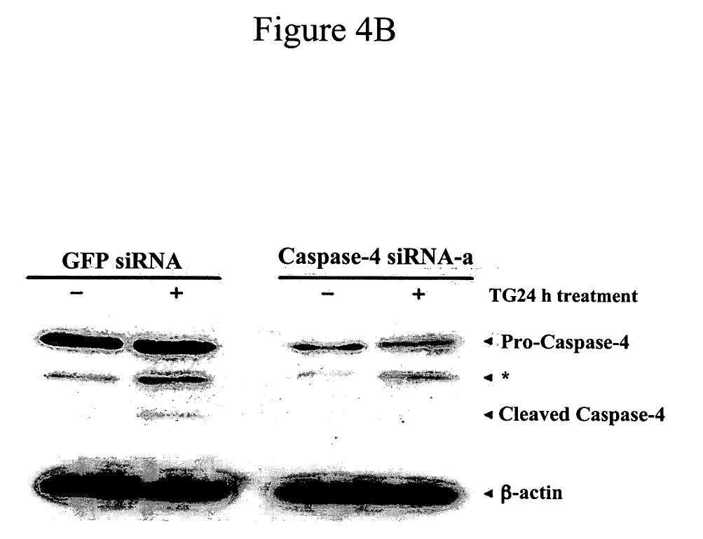

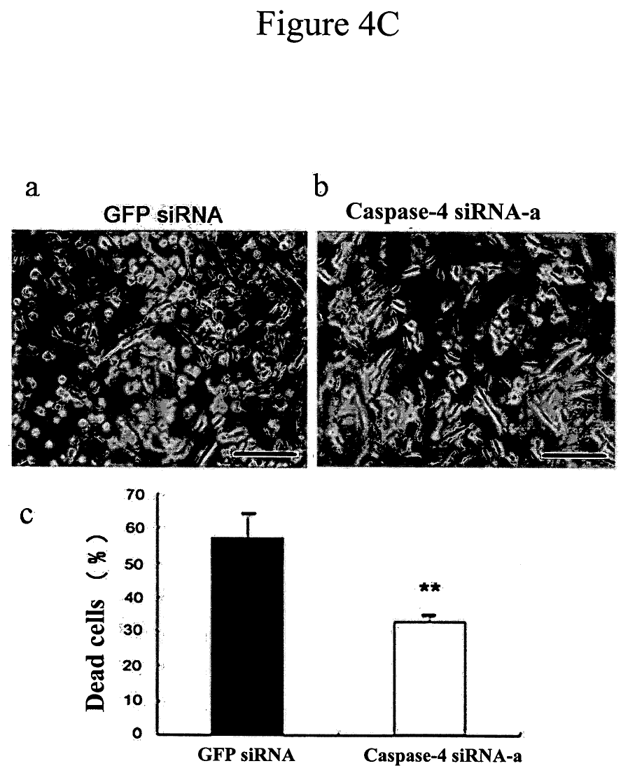

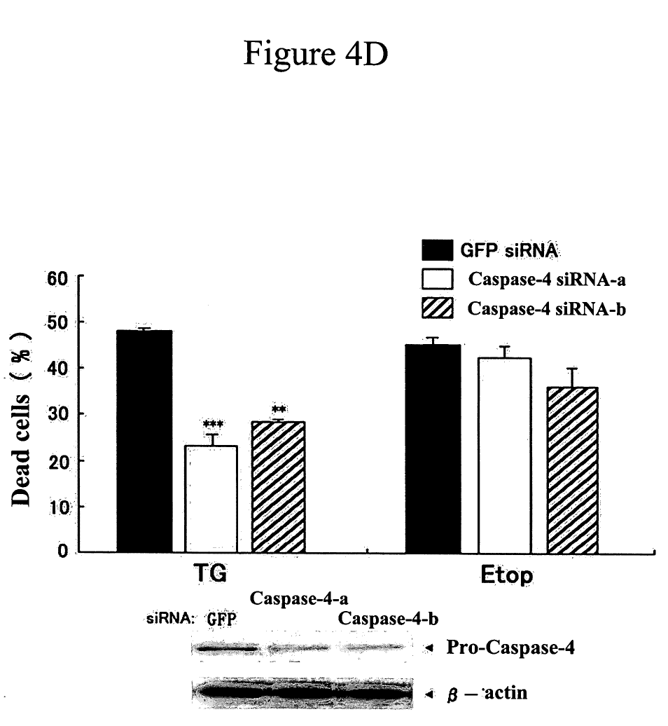

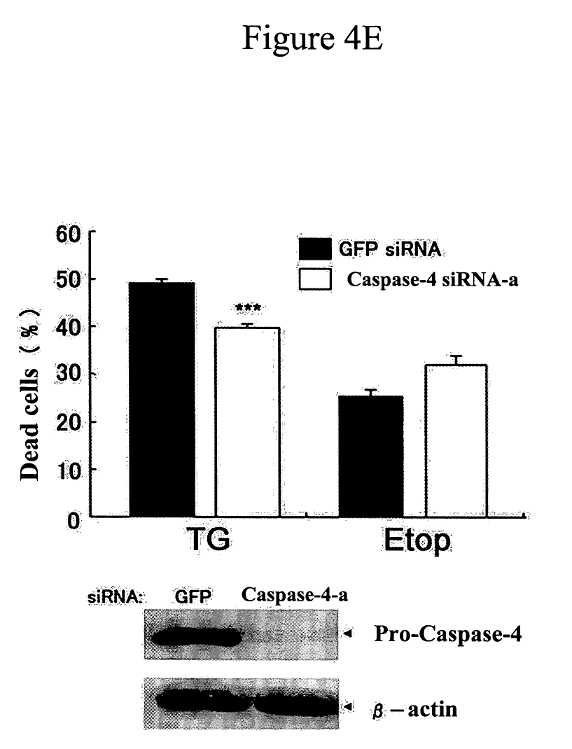

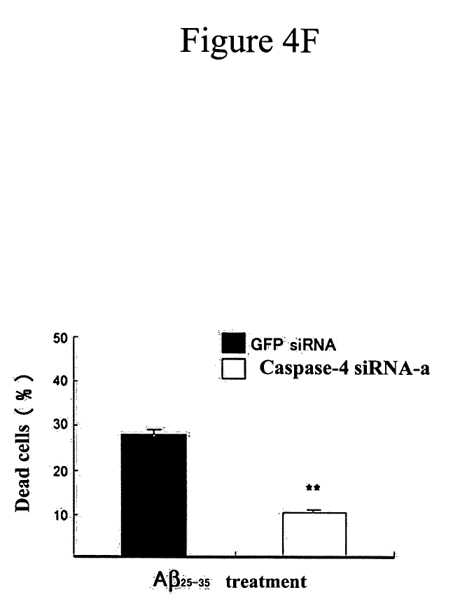

Cultured SK-N-SH cells were fixed by maintaining in 0.1 M phosphate buffer containing 4% by weight of paraformaldehyde for 2 hours at 4° C. Fixed cells were then incubated with the anti-caspase-4 monoclonal antibody or with the anti-β-actin antibody as a control, and thereafter incubated with Alexa588-conjugated mouse IgG antibodies (manufactured by Molecular Probes). Endogenous caspase-4 in stained cells were then observed under a confocal microscope (trade name: LSM510, manufactured by Carl Zeiss), to evaluate efficacy of RNAi (RNA interference). The results are shown in FIG. 4A.

As a result, as shown in FIG. 4A, it is found that the amount of caspase-4 was decreased by 60 hours of incubation after transfection with the siRNA against caspase-4 in the transfected cells, but immunoreactivity of caspase-4 was not affected by transfection with GFP siRNA as compared with that in untransfected cells.

In addition, SK-N-SH cells after the culturing were incubated in αMEM (manufactured by Invitrogen) containing 10% by weight of fetal bovine serum at 37° C. under 5% by volume of CO₂, with or without 0.5 μM thapsigargin for 40 hours. A cell extract was obtained from thapsigargin-treated SK-N-SH cells obtained after incubation in the same manner as in Example 2, and thereafter the cell extract (corresponding to 10 μg of protein) was subjected to 12% by weight SDS-PAGE. After SDS-PAGE, proteins on the gel were transferred to a PVDF membrane (manufactured by Millipore). The membrane was blocked with 5% by weight of BSA. The resulting membrane was incubated with a primary antibody (anti-caspase-4 antibody, or anti-β-actin antibody (Cat. No. 100, manufactured by Pharmingen)), and thereafter incubated with an HRP-conjugated secondary antibody. Caspase-4 or β-actin was then detected with an ECL detection system (manufactured by Amersham-Pharmacia). The results are shown in FIG. 4B.

As a result, as shown in FIG. 4B, it is found that the amount of caspase-4 was decreased by siRNA against caspase-4. It is also found that treatment with thapsigargin for 24 hours yielded lower level of cleaved-caspase-4 in the cells transfected with caspase-4-siRNA than in the cells transfected with GFP siRNA, as shown in FIG. 4B.

Furthermore, endoplasmic reticulum stress-induced apoptosis in a cell when caspase-4 level is reduced by siRNA was examined by observing morphological changes in the thapsigargin-treated SK-N-SH cells. The results are shown in FIG. 4C.

Cell death was assessed on the basis of morphological changes. As a result, as shown in Panel “c” of FIG. 4C, it is found that about 60% of untransfected cells resulted in death by treatment with thapsigargin for 40 hours. Here, cell death in the cells transfected with caspase-4 siRNA was significantly different from that in the control (cells transfected with GFP siRNA) (p<0.01).

In addition, as shown in FIG. 4C, it is found that the extent of cell death is unaffected by transfection with siRNA against GFP, but that only about 30% of the cells result in death after being transfected with caspase-4 siRNA and exposed to the same stimulation with thapsigargin.

The amount of cleaved caspase-4 shown in FIG. 4B showed good correlation with the extent of cell death shown in FIG. 4C. Thus, incomplete inhibition of cell death by transfection with caspase-4 siRNA was probably due to residual activity of caspase-4. These results indicate that cells with decreased expression of caspase-4 become more resistant to endoplasmic reticulum stress-induced cell death.

Furthermore, for the thapsigargin-treated SK-N-SH cells and HeLa cells treated with thapsigargin under the same conditions as the thapsigargin-treated SK-N-SH cells, the amount of MTS released from the cells was quantified in the same manner as in the above Example 2. The results are shown in FIG. 4D (SK-N-SH cells) and FIG. 4E (HeLa cells).

As a result, as shown in FIGS. 4D and 4E, the cells transfected with caspase-4 siRNA became more resistant to endoplasmic reticulum stress-induced cell death than the cells transfected with GFP siRNA. On the other hand, the efficiency of cell death induced by treatment with etoposide, which is a non-endoplasmic reticulum stress-inducer, was not affected by the transfection with caspase-4 siRNA. Here, cell death in the cells transfected with caspase-4 siRNA was significantly different from cell death in the control (cells transfected with GFP siRNA) (p<0.05). Therefore, it is suggested that caspase-4 is specifically involved in endoplasmic reticulum stress-induced cell death.

In addition, SK-N-SH cells transfected with GFP siRNA or caspase-4 siRNA were examined on Aβ-induced cell death as follows in the same manner as in Example 2. SK-N-SH cells were transfected with GFP siRNA or caspase-4 siRNA. The resulting cells were then incubated in αMEM (manufactured by Invitrogen) containing 10% by weight of fetal bovine serum at 37° C. under 5% by volume of CO₂ for 60 hours. The resulting cells were incubated with 25 μM Aβ₂₅₋₃₅ peptide in αMEM (manufactured by Invitrogen) containing 10% by weight of fetal bovine serum at 37° C. under 5% by volume of CO₂ for 40 hours. Survival ratio was then evaluated. The results are shown in FIG. 4F.

As shown in FIG. 4F, when treated with Aβ₂₅₋₃₅, SK-N-SH cells transfected with caspase-4 siRNA showed significantly lowered cell death as compared to the cells transfected with GFP siRNA. Here, cell death in the cells transfected with caspase-4 siRNA is significantly different from cell death in the control (cells tansfected with GFP siRNA) (**p<0.01). These results indicate that caspase-4 is essentially involved in Aβ-induced cell death, as well as in endoplasmic reticulum stress-induced cell death.

### EXAMPLE 5

Tissues of the pyramidal cell layer of the hippocampal CA1-2 region in patients with Alzheimer's disease were immunostained with antibody as described below, to confirm expression of caspase-4 in the pyramidal cell layer. Here, tissues of patients with other neurodegenerative disease were used as control.

Paraffin was removed from 10 μl of paraffin-embedded brain section by methanol containing 500 ml xylene and 1% by volume of H₂O₂. The resulting section was washed with phosphate buffered saline. Washed section was placed on a slide, subjected to blocking solution (composition: phosphate buffered saline 50 ml, 3% by weight of bovine serum albumin (1.5 ml), 3% by weight of goat serum (1.5 ml)), and allowed to stand at room temperature.

Thereafter, 32 μl of anti-human caspase-4 antibody (trade name: 4B9, manufactured by MEDICAL & BIOLOGICAL LABORATORIES CO., LTD) diluted with the above blocking solution by 200 to 500 times was added to the section on the slide. The section was incubated at 4° C. for two days.

The section on the slide was then washed with phosphate buffered saline at room temperature. Twenty milliliters of blocking solution and biotin-labeled secondary antibody (trade name: Vectastein ABC kit, manufactured by Funakoshi Co., Ltd.) were added to the washed section. Thereafter, the resulting mixture was incubated overnight at room temperature. Incubated section was washed with phosphate buffered saline for 5 minutes.

Twenty milliliters of blocking solution, purified avidin solution and biotinated alkaline phosphatase solution were mixed, to thereby carry out reaction for 30 minutes or more. The resulting mixture was then put onto the above section. Thereafter, the section was allowed to stand for 30 to 60 minutes at room temperature.

The section was then washed three times or more with phosphate buffered saline for 5 minutes, and finally washed with 100 mM Tris-hydrochloric acid buffer. Chromogenic solution (50 ml of 100 mM Tris-hydrochloric acid buffer, one DAB TABLET, 33 μl of H₂O₂) was added to the section. Thereafter, the section on the slide was washed with Tris-hydrochloric acid buffer, and observed by microscopy.

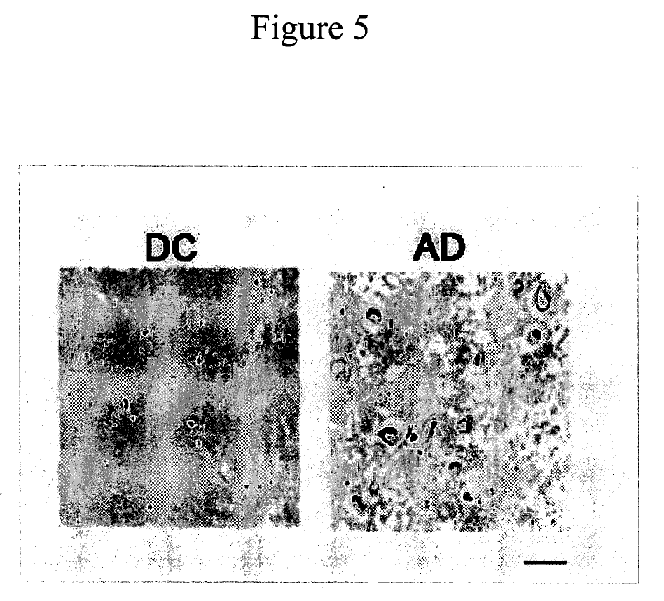

As a result, as shown in FIG. 5, it is found that caspase-4 is prominently expressed in the brain sections of the patients with Alzheimer's disease. Therefore, since apoptosis causes onset of Alzheimer's disease, it can be confirmed that caspase-4 is expressed when apoptosis is induced in a living body.

### SEQUENCE LISTING FREE TEXT

SEQ ID NO: 3 is a sequence of a sense strand of siRNA-a against caspase-4 gene.

SEQ ID NO: 4 is a sequence of an antisense strand of siRNA-a against caspase-4 gene.

SEQ ID NO: 5 is a sequence of a sense strand of siRNA-b against caspase-4 gene.

SEQ ID NO: 6 is a sequence of an antisense strand of siRNA-b against caspase-4 gene.

SEQ ID NO: 7 is a sequence of a sense strand of siRNA against GFP gene.

SEQ ID NO: 8 is a sequence of an antisense strand of siRNA against GFP gene.

SEQ ID NO: 9 is a sequence of a tetrapeptide WHED (Trp-His-Glu-Asp) which is specifically recognized by caspase-4.

### EQUIVALENT

The present invention may be embodied in other specific forms without departing from the spirit or essential characteristics thereof. The present invention is therefore to be considered in all respects as illustrative and not restrictive. The scope of the invention is indicated by the appended claims rather than by the foregoing description. Furthermore, all changes which come within the meaning and range of equivalency of the claims are therefore intended to be embraced therein.

1. A method for evaluating a substance capable of affecting endoplasmic reticulum stress- and/or amyloid β-induced apoptosis, which comprises the step of contacting a substance to be tested with a cell expressing pro-caspase-4, thereby examining behavior mediated by caspase-4 in the cell, in the presence of a substance causing endoplasmic reticulum stress or amyloid-β.

2. The evaluation method according to claim 1, wherein said cell expressing pro-caspase-4 is a cell selected from the group consisting of SK-N-SH cell, HeLa cell, HepG2 cell, SY-SY cell and a transfected cell into which a nucleic acid encoding pro-caspase-4 is introduced.

3. The evaluation method according to claim 1, wherein said cell expressing pro-caspase-4 is a transfected cell obtained by introducing a nucleic acid encoding pro-caspase-4 into HEK293 cell or HEK293T cell.

4. The evaluation method according to claim 1, which comprises the steps of:
(1) contacting a substance to be tested with a cell expressing pro-caspase-4,
(2) culturing the cell obtained in the above step (1) in the presence of a substance causing endoplasmic reticulum stress or amyloid-13, and
(3) examining changes specific to apoptosis, for the cell obtained after carrying out the step (2).

5. The evaluation method according to claim 1, which comprises the steps of:
(A) culturing a cell expressing pro-caspase-4, in the presence of a substance to be tested and a substance causing endoplasmic reticulum stress or amyloid-β, and
(B) examining changes specific to apoptosis, for the cell obtained after carrying out the step (A).

6. The evaluation method according to claim 1, wherein the presence or absence of changes in behavior caused by the presence of a substance to be tested is determined in the presence of a substrate of caspase-4.

7. The evaluation method according to claim 1, wherein said substance to be tested is a substance obtained by carrying out the steps of:
a) screening a substance binding to pro-caspase-4,
b) contacting the substance obtained by the above step a) with pro-caspase-4, in the presence of a substance causing endoplasmic reticulum stress or amyloid-β, to thereby screen a substance generating cleaved products thereof, and
c) preparing a substance to be tested, based on a cleavage site of the substance obtained in the above step b) by caspase-4, as a derivative containing a cleavage site region of the substance.

8. The evaluation method according to claim 1, wherein said substance to be tested is a substance selected from the group consisting of a derivative of a substrate of caspase-4, a mimic of the substrate, and a substance capable of binding to caspase-4.

9. The evaluation method according to claim 1, wherein said behavior is at least one event selected from the group consisting of karyopyknosis, fragmentation of a chromosome, shrinkage of a cell, release of MTS, cleavage of pro-caspase-4, release of lactate dehydrogenase, and generation of a product resulting from a substrate of caspase-4.

10. A kit for evaluating a substance capable of affecting endoplasmic reticulum stress- and/or amyloid β-induced apoptosis, for carrying out the evaluation method of any one of claims 1 to 9, which comprises a cell expressing pro-caspase-4, a substance causing endoplasmic reticulum stress or amyloid-β, and a reagent suitable for contacting a substance to be tested with the cell.

11. A pharmaceutical composition, which comprises as an active ingredient, a substance evaluated by the evaluation method of any one of claims 1 to 9, wherein said substance is a substance capable of affecting endoplasmic reticulum stress- and/or amyloid β-induced apoptosis.

12. The pharmaceutical composition according to claim 11, wherein said substance is an siRNA specific to pro-caspase-4 or caspase-4, or an antisence nucleic acid against a nucleic acid encoding caspase-4.


SEQUENCE LISTING (7 sequences)

SEQ ID NO: 1 - DNA, 1306 bp, Homo sapiens
  CDS (40)..(1173)
  ref <301>: AUTHORS: Hitomi J, Katayama T, Eguchi Y, Kudo T, Taniguchi M, Koyama Y, Manabe T, Yamagishi S, Bando Y, Imaizumi K, Tsujimoto Y, Tohyama M.
  ref <302>: TITLE: Involvement of caspase-4 in endoplasmic reticulum stress-induced apoptosis and Abeta-induced cell death
  ref <303>: JOURNAL: The Journal of Cell Biology
  ref <304>: VOLUME: 165
  ref <305>: ISSUE: 3
  ref <306>: PAGES: 347-356
  ref <307>: DATE: 2004-05-10
cttttccaacgctgtaaaaaaggacagaggctgttccctatggcagaaggcaaccacagaaaaaagccacttaaggtgttggaatccctgggcaaagatttcctcactggtgttttggataacttggtggaacaaaatgtactgaactggaaggaagaggaaaaaaagaaatattacgatgctaaaactgaagacaaagttcgggtcatggcagactctatgcaagagaagcaacgtatggcaggacaaatgcttcttcaaaccttttttaacatagaccaaatatcccccaataaaaaagctcatccgaatatggaggctggaccacctgagtcaggagaatctacagatgccctcaagctttgtcctcatgaagaattcctgagactatgtaaagaaagagctgaagagatctatccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctggaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattgaaaatggaagccacaagcagcccagccctccttaatcaacttcaaggagcaccttcattagtacagcttgcatatttaacattttgtatttcaataaaagtgaggaaaaaaaaaaaaaaaaaaaaaaaaaaaaa

SEQ ID NO: 2 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 3 - DNA, 21 bp, Artificial Sequence
  synthetic sense strand of siRNA for caspase-4 gene
guggccucuucacagucautt

SEQ ID NO: 4 - DNA, 21 bp, Artificial Sequence
  synthetic antisense strand of siRNA for caspase-4 gene
augacugugaagaggccactt

SEQ ID NO: 5 - DNA, 23 bp, Artificial Sequence
  synthetic sense strand of siRNA for GFP gene
  modified_base (1)..(1): p
ugggcuacguccaggagcgcacc

SEQ ID NO: 6 - DNA, 22 bp, Artificial Sequence
  synthetic antisense strand of siRNA for GFP gene
  modified_base (1)..(1): p
uugcgcuccuggacguagccuu

SEQ ID NO: 7 - protein, 4 aa, Artificial Sequence
  a synthetic sequence of tetrapeptide WHED (Trp-Glu-His-Asp) which is specifically recognized by caspase-4
WHED